(19) United States
(12) Patent Application Publication
(10) Pub. No.: US 2023/0321517 A1
(43) Pub. Date: Oct. 12, 2023

(54) Method and Apparatus for Strengthening or Increasing Mass of a Pelvic Floor Muscle

(71) Applicant: S-Alpha Therapeutics, Inc., Seoul (KR)

(72) Inventors: Seung Eun Choi, Seoul (KR); Tae Hyun Kim, Seoul (KR)

(73) Assignee: S-Alpha Therapeutics, Inc., Seoul (KR)

(21) Appl. No.: 18/025,267

(22) PCT Filed: Sep. 8, 2021
(86) PCT No.: PCT/KR2021/012247

§ 371 Date: Mar. 8, 2023

Related U.S. Application Data
(60) Provisional application No. 63/075,414, filed on Sep. 8, 2020.

(51) Int. Cl. A63B 71/06; A63B 24/00

(52) U.S. Cl. CPC A63B 71/0622; A63B 24/0087; A63B 2230/06; A63B 2071/065

(57) ABSTRACT

The present disclosure provides various methods and systems for strengthening or increasing mass of a pelvic floor muscle in a subject by providing, by an electronic device to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising one or more first instructions for the subject to follow.

### TECHNICAL FIELD

The present disclosure relates to digital therapeutics (hereinafter referred to as DTx) intended for strengthening or increasing mass of a pelvic floor muscle in a subject.

### BACKGROUND ART

Pelvic floor muscle training commonly provided at hospitals uses biofeedback medical apparatus which bases on electromyograph (EMG) to monitor the movement of pelvic floor muscle and correct wrong movements. By attaching or inserting a sensor to the abdomen and anal canal, and measuring the EMG of pelvic floor muscle with the sensor, the movement of pelvic floor muscle can be monitored. The training usually lasts for an hour-long session while the patient is visiting the hospital and should be conducted over a long period. However, the aforementioned biofeedback pelvic floor muscle training apparatus cannot be easily used by patients without visiting hospitals and it is very costly.

### DISCLOSURE OF INVENTION

### Solution to Problem

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

Methods and apparatuses are described herein for strengthening or increasing mass of a pelvic floor muscle (PFM) in a subject. For example, an apparatus may generate one or more instructions for PFM treatment. The PFM instructions may induce, based on a patient's performance of the PFM instructions, at least one physiological response to strengthen and/or increase the mass of the PFM of the patient. In some embodiments the patient may require post-operative rehabilitation (e.g., after an anorectal anomaly reconstruction operation). The biological response(s) may include at least one of growth hormone/insulin-like growth factor-1 (GH/IGF-1) secretion, non-ischemic status, enteric nerve system (ENS) maturation and GLP2 secretion. The physiological response(s) may include at least one of PFM myogenesis, anal sphincter function recovery, ganglion regeneration and GI motility adaptation. The one or more PFM instructions may comprise Exercise/Life style, Sitz bath, pelvic splanchnic nerve stimulation (PSNS)/PFM exercise, and vagal nerve stimulation (VNS)/gastrointestinal (GI) exercise modules. The apparatus may then provide the patient the one or more PFM instructions.

### BRIEF DESCRIPTION OF DRAWINGS

FIG. 1A and FIG. 1B are diagrams illustrating an example mechanism of action (MOA) for strengthening or increasing mass of a pelvic floor muscle;

FIG. 2 is a diagram illustrating the standard of care in anorectal anomaly reconstruction operation and rehabilitation;

FIG. 3 is a diagram illustrating GI-post-operation recovery. The flow chart illustrates an exemplary process for pediatric post-operative rehabilitation of a subject having a lower gastrointestinal-operable-congenital anomaly;

FIG. 4 is a diagram illustrating an example changes in contribution of the patient and guardian affecting adherence in accordance with the patient's growth;

FIG. 5 is a diagram illustrating an example Exercise/Life style module;

FIG. 6 is a diagram illustrating an example Sitz bath module;

FIG. 7 is a diagram illustrating an example PSNS/PFM exercise;

FIG. 8 is a diagram illustrating an example VNS/GI exercise module;

FIG. 9 is a table showing VN innervation and stimulation methods;

FIG. 10 is a diagram illustrating Interactive feedback between PFM application and users;

FIG. 11 is a diagram illustrating an examples of instruction input and practice monitoring of PFM application;

FIG. 12 is a diagram illustrating the embodiment of a PFM app;

FIG. 13 is a diagram illustrating a mechanism of action for how various modules of the present disclosure can contribute to post-operative rehabilitation of a subject having a lower gastrointestinal-operable-congenital anomaly;

FIG. 14 is a system diagram illustrating an example device that can be used for PFM treatment;

FIG. 15 is a diagram showing an exemplary feedback loop for a digital apparatus and an digital application for post-operative rehabilitation of subjects with a lower gastrointestinal-operable-congenital anomaly according to one embodiment of the present disclosure;

FIG. 16 is a flowchart illustrating exemplary operations in a digital application for post-operative rehabilitation of subjects with a lower gastrointestinal-operable-congenital anomaly according to one embodiment of the present disclosure;

FIG. 17 is a diagram showing an exemplary hardware configuration of the digital apparatus for post-operative rehabilitation of subjects with a lower gastrointestinal-operable-congenital anomaly according to one embodiment of the present disclosure;

FIG. 18 is a flow chart illustrating an exemplary system for post-operative rehabilitation of subjects with a lower gastrointestinal-operable-congenital anomaly, the system comprising an administrative portal (e.g., Administrator's web), a healthcare provider portal (e.g., Doctor's web) and a digital apparatus configured to execute a digital application (e.g., an application or ‘app’) for post-operative rehabilitation of subjects with a lower gastrointestinal-operable-congenital anomaly;

FIG. 19 is a flow chart illustrating an exemplary execution flow for a login verification during a splash process at the starting of a digital application of the present disclosure;

FIG. 20 is a flow chart illustrating an exemplary execution flow for a prescription verification during a splash process at the starting of a digital application of the present disclosure;

FIG. 21 is a flow chart illustrating an exemplary execution flow for a healthcare provider portal in a system of the present disclosure;

FIGS. 22A-H depict (A) an exemplary dashboard of a healthcare provider portal, (B) an exemplary patient tab in a healthcare provider portal, the patient tab displaying a list of patients, (C) an exemplary patient tab in a healthcare provider portal, the patient tab displaying detailed information on a given patient, (D) an exemplary patient tab in a healthcare provider portal for adding a new patient, (E) an exemplary patient tab in a healthcare provider portal for editing information of an existing patient, (F) an exemplary patient tab in a healthcare provider portal that displays detailed prescription information for a given patient, and (G-H) an exemplary patient tab in a healthcare provider portal for editing prescription information for a given patient;

FIG. 23 is a flow chart illustrating an exemplary execution flow for an administrative portal in a system of the present disclosure;

FIGS. 24A-H depict (A) an exemplary dashboard of an administrative portal, (B) an exemplary doctor tab in an administrative portal, the doctor tab displaying a list of doctors, (C) an exemplary doctor tab in an administrative portal, the doctor tab displaying a list of patients being cared for by a given doctor, with patient-identifying information redacted (*), (D) an exemplary doctor tab in an administrative portal for adding a new doctor, (E) an exemplary doctor tab in an administrative portal for editing information of an existing doctor, (F) an exemplary patient tab in an administrative portal that displays information for one or more patients, wherein sensitive in formation is redacted, (G) an exemplary patient tab in an administrative portal that displays detailed patient or prescription information for a given patient, and (H) an exemplary patient tab in an administrative portal that displays detailed prescription information for a given patient; and

FIG. 25 depicts an exemplary interfaces for the start screen, voluntary activity, induction of action, and provision of rewards.

FIG. 26 depicts an exemplary game list.

FIGS. 27-32 depict exemplary embodiments for body sensing during the activities.

FIGS. 33-36 depict exemplary screens for skin massage, sitz bath, activity and sleep.

While the above-identified drawings set forth presently disclosed embodiments, other embodiments are also contemplated, as noted in the discussion. This disclosure presents illustrative embodiments by way of representation and not limitation. Numerous other modifications and embodiments may be devised by those skilled in the art which fall within the scope and spirit of the principles of the presently disclosed embodiments.

### MODE FOR THE INVENTION

One function of the pelvic floor muscle is to support organs that are located in the pelvis area. Urinary and fecal incontinence, prolapses, infections, inflammations and pain in the vaginal and rectum area, decreased sexual pleasure, and other conditions, can all result from dysfunctional pelvic floor muscles. Dysfunction in the pelvic floor muscles can be the result of a variety of factors, including genetic or developmental defects, hormonal changes, aging, heavy lifting, excessive coughing, and disease. Exercises employing pelvic floor muscle clenching have been developed to assist with improving pelvic floor muscle strength, however these methods have limited success since it is difficult to quantify if the exercise is being performed correctly by the subject at all times. Furthermore, existing methods fail to consider the underlying mechanism of action for the particular condition causing dysfunction of the pelvic floor muscle in the subject, which can aid a healthcare provider in determining the most effective course of treatment.

The present disclosure provides various methods and systems for strengthening or in creasing mass of a pelvic floor muscle in a subject by providing, by an electronic device to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising one or more first instructions for the subject to follow. Certain embodiments of the present disclosure may comprise deducing a mechanism of action (hereinafter referred to as MOA) in a subject having pelvic floor muscle dysfunction through a literature search and expert reviews of basic scientific articles and related clinical trial articles to find the mechanism of action in pelvic floor muscle dysfunction, and establishing a therapeutic hypothesis and a digital therapeutic hypothesis for strengthening or in creasing mass of a pelvic floor muscle in a subject and treating the pelvic floor muscle dysfunction based on these findings. The present disclosure also relates to a rational design of a digital application for clinically verifying a digital therapeutic hypothesis for pelvic floor muscle dysfunction in a subject and realizing the digital therapeutic hypothesis for digital therapeutics.

In some cases, dysfunction of the pelvic floor muscle can be caused, at least in part, by a meningocele, which refers to a birth defect where there is a sac protruding from the spinal column. Hirschsprung's disease (HD or HSCR) refers to a birth defect in which nerves are missing from parts of the intestine. The incidence ratio of the HD or HSCR is 1:5000 birth. Anorectal malformations (ARM) refer to birth defects or problems that happen as an unborn baby is developing during pregnancy. With this defect, the anus and rectum do not develop properly. The incidence ration of the ARM is 1:5000 birth

The degree of meningocele differs from person to person, hence its treatment differs. The treatment of Herschsprung's disease may include pull-through surgery and Ostomy surgery. Anorectal malformation may use different operations depending on the incidence—low, middle and high. This basically aims to create a new anus.

After the treatments, surgery, or operations (e.g., gastrointestinal anomaly reconstruction operation), patients may experience post-operative complication. For example, fecal incontinence, stricture, and constipation occur in the patient.

The purpose of rehabilitation is to prevent anal strictures and fecal incontinence after reconstruction operation, thereby overcoming the post-operative complication. The re habilitation measures or methods may include: (1) gastrointestinal (GI) training, for example, visiting hospital/clinic, and enforcing sphincter using the trocar (gradually in creasing size) to prevent stricture; and (2) anal biofeedback.

Anal sphincter complex comprises inner tube (i.e. internal anal sphincter) and outer tube (i.e. external anal sphincter), each corresponding to smooth muscle and skeletal muscle. It was reported that the smooth muscle of internal anal sphincter also re generates in adult and mesenchymal stem cell (MSC) relates to the smooth muscle re generation. Active skeletal muscle exercise, nutrition (e.g., protein, amino acid uptake), and adequate sleep induce GH, IGF-1 secretion. The humoral factors induce SMC's differentiation and myogenesis on the surgery area. As a result, stricture can be prevented.

Pelvic floor muscles are the layer of muscles that support the pelvic organs and span the bottom of the pelvis. The pelvic organs are the bladder and bowel in men, and bladder, bowel and uterus in women.

Pelvic floor muscle training commonly provided at hospitals uses biofeedback medical apparatus which bases on electromyograph (EMG) to monitor the movement of pelvic floor muscle and correct wrong movements. By attaching or inserting a sensor to the abdomen and anal canal and measuring the EMG of pelvic floor muscle with the sensor, the movement of pelvic floor muscle can be monitored. When a muscle contracts or relaxes, a nurse specialist assists to help the patient perform the correct muscle training. The training usually lasts for an hour-long session and should be conducted over a long period. However, there exist some constraints such that a patient has to visit the hospital for every single training session.

Morpheus® of The Prometheus Group is a medical apparatus used at hospitals for pelvic floor muscle rehabilitation. Morpheus supports Complex Urodynamics, Anorectal Manometry with Paradoxical sEMG, and Multicompartment Pelvic Floor Ultrasound Imaging and others to track the movement of pelvic floor muscle. It works by inserting a sensor to the patient's anorectal.

Measures to enhance the strength of pelvic floor muscle have developed in a more private and user-centered way. Pelvictrainer of Meden-Inmed works by piezoelectric sensing of pelvic floor muscle movement, hence a probe is not needed and the patient need not to take his/her clothes off.

The problems of the aforementioned biofeedback pelvic floor muscle training apparatus are that it cannot be easily used without visiting hospitals and it is very costly.

Although their purposes are not directly forward the rehabilitation after post-op re construction operation, the cases below describe some examples of developing rehabilitation methods and apparatuses.

Smart Shorts of VylyV Lab is Kegel exercise equipment. Male patients tend to not actively perform exercises, although they are aware of the need of pelvic floor muscle training for their potency, because it is difficult for them to accurately recognize the contraction and relaxation of the muscles. Smart Shorts of VylyV Lab sends the muscle movement data to the user's smartphone through a built-in sensor of a short-pants-form wearable device and a wireless transmitter. The smartphone application that has received the data displays the degree of muscle contraction and relaxation and provides relevant games using the movement data of the penis.

Emy of Fizimed is Kegel exercise equipment for women. Similar to the equipment of VylyV, it provides personal apparatus and smartphone application. Emy measures the contraction and relaxation of the muscles by inserting the equipment inside a woman's vagina. Because the equipment is inserted inside a vagina, women patients can perform training/exercises with their clothes on, check relevant data of pelvic floor muscle con traction and relaxation through the smartphone application, and play relevant games.

Both of the equipment above has helped pelvic floor muscle exercise to be performed in a different way than the original hospital-centered way. Also, by engaging sensing technology, it allows patients to check their data themselves.

An apparatus developed to assist pelvic floor muscle exercise (‘VAGINA CONTRACT TRAINING SYSTEM AND METHOD THEREOF (KR 10-1879680 B1)’) is designed for the user to insert a vibrator inside her vagina and control the level of difficulty and vibrating patterns via the smartphone. ‘INTEGRATED DIAGNOSIS AND TREATMENT DEVICE FOR URINARY INCONTINENCE AND SEXUAL DYSFUNCTION THROUGH CONNECTION TO SMART PHONE (KR 10-1325581 B1)’ measures a user's systolic pressure and duration time of pelvic floor muscle, perineal muscle, anal sphincter via a main body which has a pressure sensor and a vibrating function, and displays the data to the application installed in the user's smartphone. Both technologies measure pelvic floor muscle movement by inserting pressure indicators and/or vibrators inside the body and display the data on smartphones to present to the user.

Nerve systems directly engaged with intestines and anorectal to affect relevant muscles are the parasympathetic nerves of autonomic nervous system, especially, vagal nerve (VN), pelvic splanchnic nerve (PSN), and enteric nerve system (ENS).

Parasympathetic effector system serves to: (1) dilate blood vessels leading to the GI tract, increasing the blood flow; and (2) accelerate peristalsis, mediating digestion of food and indirectly the absorption of nutrients.

In order to ease post-operative complication such as fecal incontinence, stricture and constipation, acceleration of bowel movement and peristalsis are needed. This may be accomplished via VN/PSN stimulation and ENS maturation. In order to rehabilitate bowel and anorectal functions after the congenital anomaly reconstruction operation, effective muscle function may need to be induced by restraining ischemic state in order for stem cell to be divided into myoblast based on Exeercise/Life style, Sitz bath module such as exercise, skeletal exercise, sufficient sleep, sitz bath or the like. In additional, effective action of the nervous system may be induced by stimulating vagal nerve and pelvic splanchnic nerve of particularly parasympathetic nerves among the autonomic nervous system which engages in maintaining balance in terms of bowel movement and sphincter control. Specifically, this may be accomplished by the related PSNS/PFM exercise, VNS/GI exercise module such as vagal nerve stimulation (VNS) (e.g., massage, feeding, etc.), pelvic splanchnic nerve (PSN) stimulation (e.g., biofeedback training, etc.), or the like.

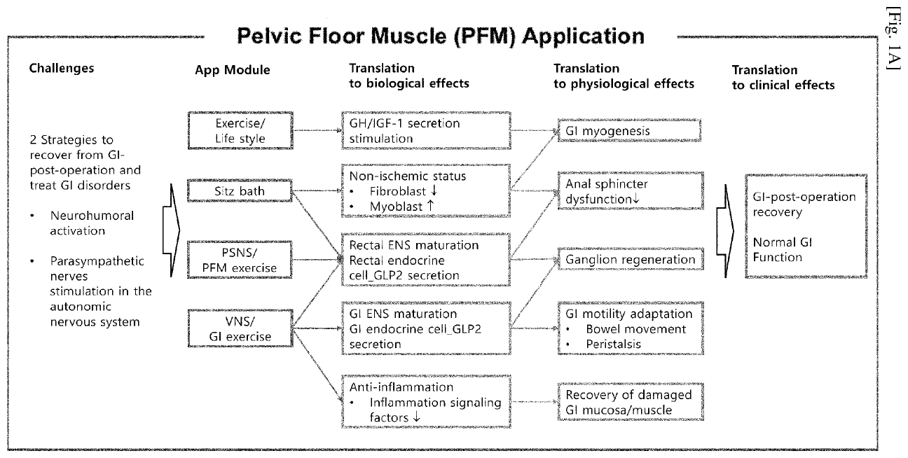

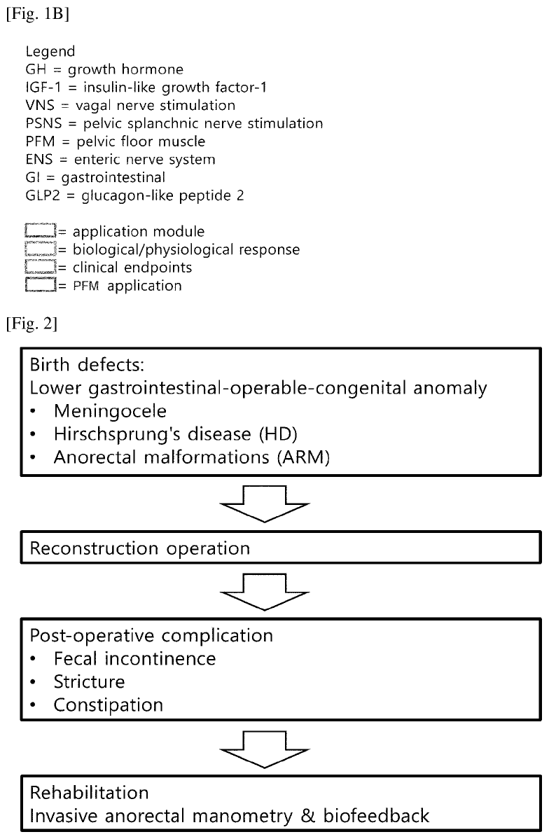

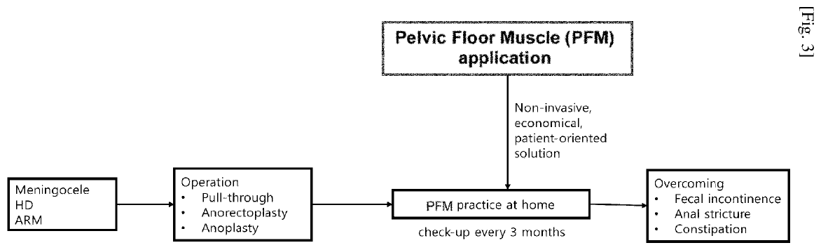

FIG. 1A and FIG. 1B are diagrams illustrating an example mechanism of action (MOA) for post-operative rehabilitation of children with lower gastrointestinal-operable-congenital anomaly.

As illustrated in FIG. 1A and FIG. 1B, the application modules represent a series of behaviors that a user practices through the instructions on PFM application, and the translation to biological/physiological effects present the biological/physiological response and its results in accordance with the user's behaviors. Exercise/Life style module may instruct the exercises suitable for GH/IGF-1 secretion. Sitz bath exercise module accelerates blood circulation by warm temperature and makes non-ischemic status for myoblast differentiation. As a result of the myoblast, anal sphincter dysfunction will be reduced. PSN/PFM exercise module may include, but are not limited to, anorectal ENS maturation through PSN and PFM exercise. They also accelerate new anorectal ganglion regeneration. VNS/GI exercise module may induce GI ENS maturation and stimulate GI endocrine cells. As a result of VNS/GI exercise, GI motility and GLP2 secretion may be activated. They also accelerate new GI ganglion regeneration. Hence, the PFM application may help the child's rehabilitation after the reconstruction operation through user's instruction-practice feedback.

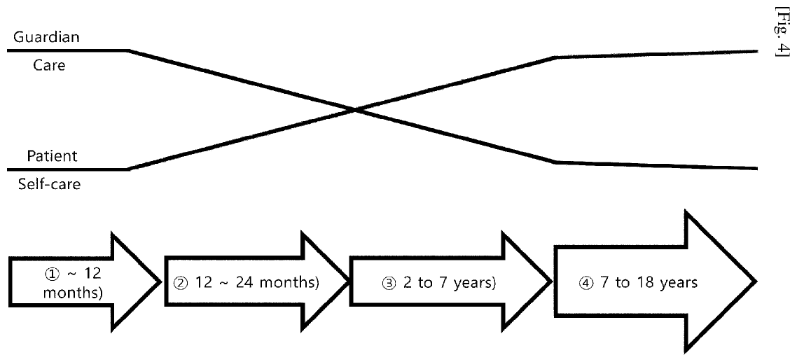

FIG. 2 is a diagram illustrating the standard of care in anorectal anomaly reconstruction operation and rehabilitation, which may be used in combination with any of other embodiments described herein.

FIG. 4 is a diagram illustrating an example changes in contribution of the patient and guardian affecting adherence in accordance with the patient's growth, which may be used in combination with any of other embodiments described herein.

Although anorectal reconstruction operation takes place soon after one's birth, its re habilitation process persists until the patient becomes an adult. Patient's active participation and adherence being necessary for therapeutic efficacy in terms of digital therapeutics, the criteria for active participation and adherence may vary significantly in accordance with the patient's 4 growth stages: 1.) birth to 12 months, 2.) 12 to 24 months, 3.) 2 to 7 years, 4.) 7 to 20 years.

In fact, during 1.), 2.) stage, guardian's active involvement may be more important than patient's self care. However, as the patient grows, the importance of self care may become greater than the guardian's role.

Embodiments described below provide specific instructions for each growth stages in the order of 1.), 2.), 3.), and 4.).

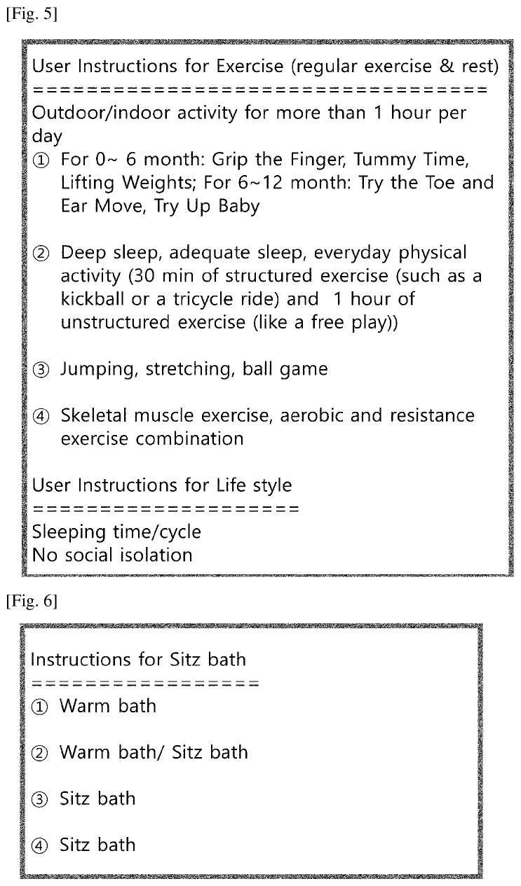

FIG. 5 is a diagram illustrating an example Exercise/Life style module that includes one or more exercises inducing growth hormone/insulin-like growth factor-1/(GH/IGF-1) secretion, which may be used in combination with any of other embodiments described herein.

Exercise instructions provided in accordance with a child's or patient's growth are as follows: 1.) for 0-6 month old: Grip the Finger, Tummy Time, Lifting Weights and for 6-12 month old: Try the Toe and Ear Move, Try Up Baby; 2.) deep sleep, adequate sleep, everyday physical activity (30 min of structured exercise (such as a kickball or a tricycle ride) and 1 hour of unstructured exercise (like a free play)); 3.) jumping, stretching, ball game; and 4.) skeletal muscle exercise, aerobic and resistance exercise combination. As described in FIG. 4, patient's voluntary participation is emphasized as he/she grows older.

By practicing these types of exercise instructed by Exercise/Life style module, the secretion of humoral factors such as GH/IGF-1 may be increased, which in turn accelerates differentiation of SMC to smooth muscle. This kind of acceleration of myogenesis helps muscle regeneration near intraoperative location.

FIG. 6 is a diagram illustrating an example an example Sitz bath module that accelerates blood circulation by warm temperature and makes non-ischemic status for myoblast differentiation instead of fibroblast.

In the case of warm bath/sitz bath, its duration, frequency, and temperature of water may gradually increase as the child or the patient grows. Warm bath/sitz bath may use warm water to raise temperature near the anus and dilate blood vessels, restraining ischemic state and preventing stricture of muscles around the anus.

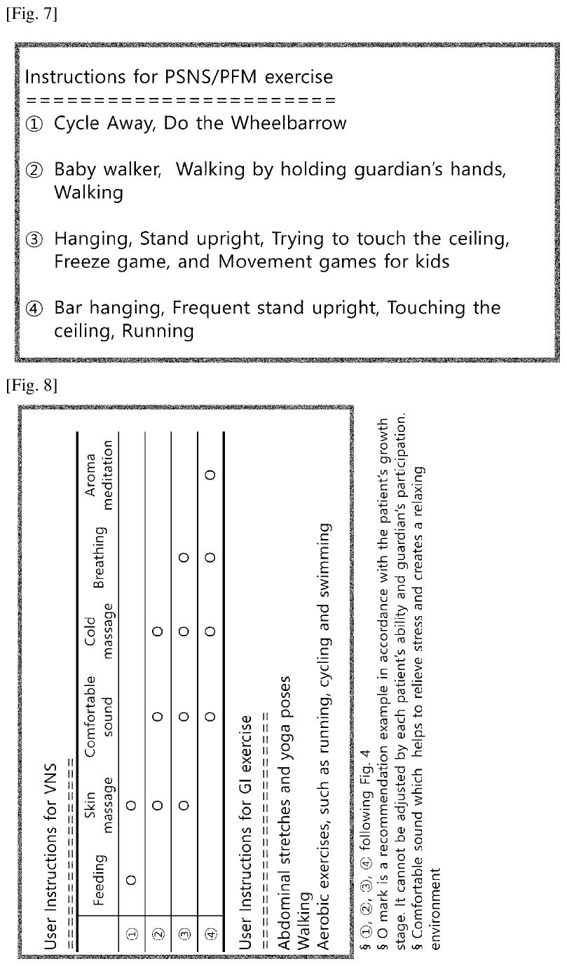

FIG. 7 is a diagram illustrating an example PSN/PFM exercise module that induces PSN stimulation and pelvic floor muscle exercise, which may be used in combination with any of other embodiments described herein.

PSNS/PFM exercise instruction may be provided differently for different categories of PSNS and PFM exercise. Each category may be further divided by a child's or patient's growth development stage into 1.), 2.), 3.), and 4.) to provide relevant instructions. On the other hand, PFM exercise may intensify anorectal muscle to alleviate fecal incontinence. GI exercise instruction may be also provided differently in accordance with the child's or patient's growth period or development.

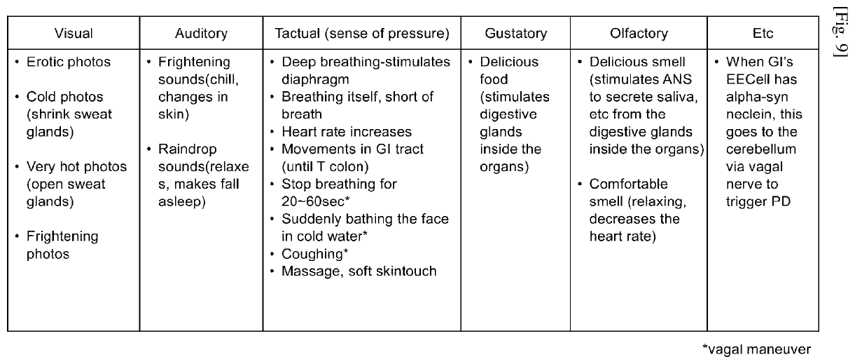

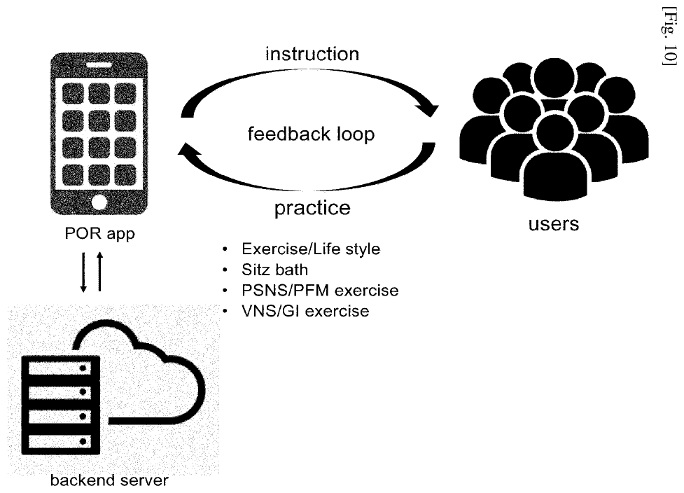

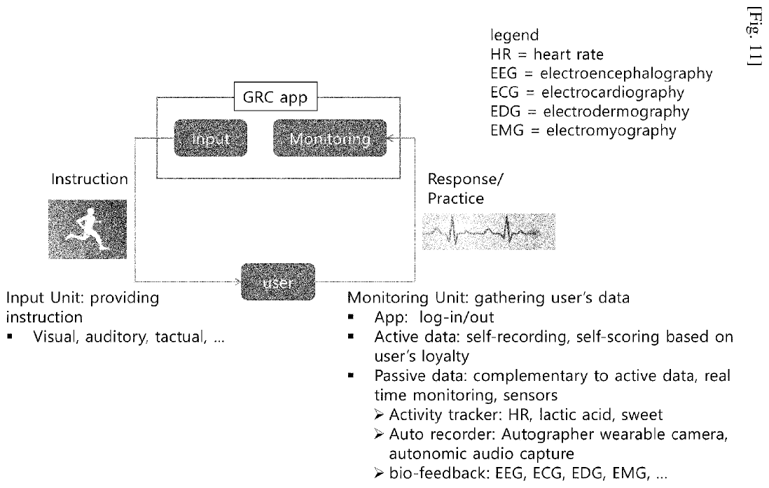

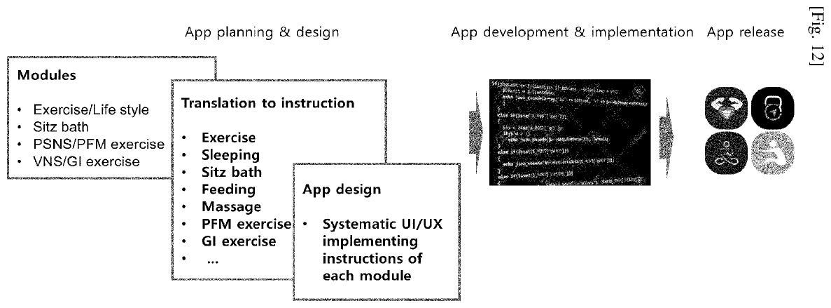

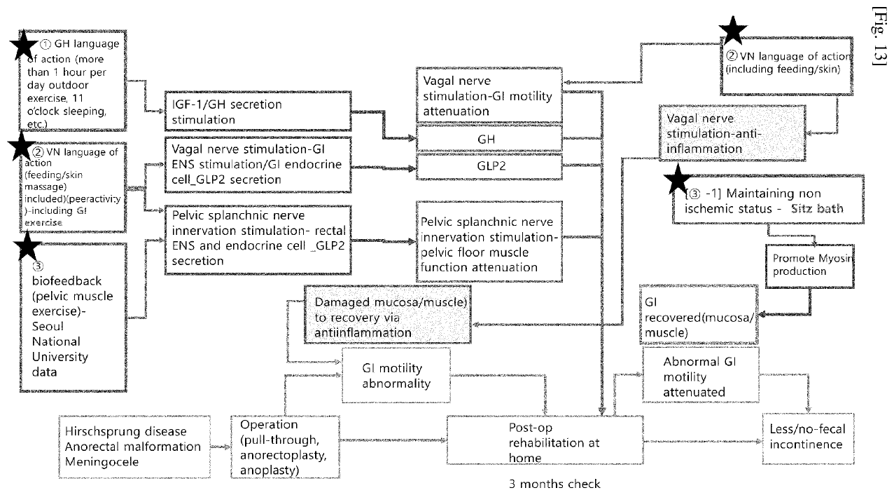

FIG. 8 is a diagram illustrating an example VNS/GI exercise module that induces vagal nerve stimulation (VNS) and gastrointestinal (GI) motility, which may be used in combination with any of other embodiments described herein.

As illustrated in FIG. 8, various VNS for appropriate age groups may accelerate GI motility and GLP2 secretion, thereby accelerating bowel movement and peristalsis to prevent constipation.

FIG. 9 is table showing VN innervation and stimulation methods, which may be used in combination with any of other embodiments described herein.

As illustrated in FIG. 9, various means are listed to stimulate the vagal nerve. The vagal nerve stimulation may be practiced, for example, by involving visual, auditory, tactual, gustatory and olfactory senses, molecules, or the like. Vagal nerve innervation instructions may include cold massage, deep breathing, abdominal breathing, listening to music, watching movies, and the like.

FIG. 10 is a diagram illustrating an example pelvic floor muscle (PFM) treatment application in communication with other entities for the post-operative rehabilitation, which may be used in combination with any of other embodiments described herein. As illustrated in FIG. 10, the PFM application may be embodied in the form of application in any digital devices. Examples of the digital devices may include, but are not limited to, a desktop computer, a laptop computer, a tablet, a smartphone, and a wireless transmit/receive unit (WTRU). A user may download the PFM application, and receive/perform the instructions via the PFM application. The user's practice or performance may give feedback to the application through various means, for example, passive data gathering using log-in/out information, user review/evaluation, sensors, etc. The instruction-practice feedback loop may increase the users' adherence to induce their constant and voluntary participation. Taking a closer look at the specific physiological responses by the PFM application, the following may be induced: Exercise/Life style, Sitz bath, PSNS/PFM exercise, and VNS/GI exercise module as a result of the instruction-practice process. The PFM application may be designed based on the MOA that induces the aforementioned physiological responses. Consequently, pediatric patients and their parents/guardians can actively participate in overcoming post-operative complication through the practiced instructions.

FIG. 11 is a diagram illustrating examples of instruction input and practice monitoring of PFM application, which may be used in combination with any of other embodiments described herein. As illustrated in FIG. 11, various combination between input and monitoring of PFM application may be used. The instruction input may be provided to a user by visual display, auditory narration, touch/vibration, or the like. The practice out can be classified to three categories: (1) application log-in/out in formation; (2) active data which are generated by user's typing or recording; and (3) passive data which are gathered by sensors. The sensors for passive data gathering may include, but are not limited to, activity trackers, auto recorders, and bio-feedback instruments.

FIG. 12 is a diagram illustrating the embodiment of PFM application, which may be used in combination with any of other embodiments described herein.

As illustrated in FIG. 12, the PFM application may provide/specify the instructions of the Exercise/Life style, Sitz bath, PSNS/PFM exercise, and VNS/GI exercise module of FIGS. 1, 5-8, and reflect them in the application design. The PFM application may be made and be launched based on the application programming that reflects the aforementioned application planning.

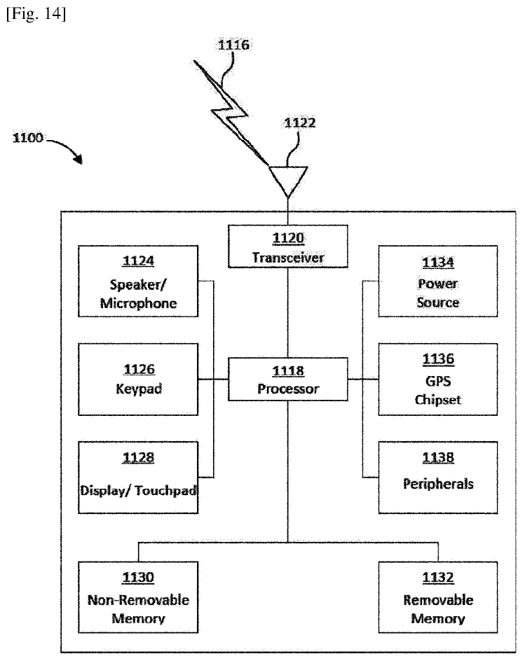

FIG. 14 illustrates an example device 1100 that can be used for PFM treatment, which may be used in combination with any of other embodiments described herein. As shown in FIG. 14, the device 1100 may include a processor 1118, a transceiver 1120, a transmit/receive element 1122, a speaker/microphone 1124, a keypad 1126, a display/touchpad 1128, non-removable memory 1130, removable memory 1132, a power source 1134, a global positioning system (GPS) chipset 1136, and/or other peripherals 1138, among others. It will be appreciated that the device 1100 may include any sub-combination of the foregoing elements while remaining consistent with an embodiment. By way of example, the device 1100 may include at least one of a mobile device, a user equipment (UE), a mobile station, a fixed or mobile subscriber unit, a subscription-based unit, a pager, a cellular telephone, a personal digital assistant (PDA), a smartphone, a laptop, a netbook, a personal computer, a wireless sensor, a hotspot or Mi-Fi device, an Internet of Things (IoT) device, a watch or other wearable, a head-mounted display (HMD), a vehicle, a drone, a medical device and applications (e.g., remote surgery), an industrial device and applications (e.g., a robot and/or other wireless devices operating in an industrial and/or an automated processing chain contexts), a consumer electronics device, a device operating on commercial and/or industrial wireless networks, and the like.

The processor 1118 may be a general purpose processor, a special purpose processor, a conventional processor, a digital signal processor (DSP), a plurality of micro processors, one or more microprocessors in association with a DSP core, a controller, a microcontroller, Application Specific Integrated Circuits (ASICs), Field Programmable Gate Arrays (FPGAs), any other type of integrated circuit (IC), a state machine, and the like. The processor 1118 may perform data processing, power control, input/output processing, sensor date processing, and/or any other functionality that enables the device 1100 to provide antiviral digital vaccine. The processor 1118 may be coupled to the transceiver 1120, which may be coupled to the transmit/receive element 1122. While FIG. 14 depicts the processor 1118 and the transceiver 1120 as separate components, it will be appreciated that the processor 1118 and the transceiver 1120 may be integrated together in an electronic package or chip.

The transmit/receive element 1122 may be configured to transmit data to, or receive data from a sever located in a medical institution or institution that manages the PFM application. For example, instructions from a doctor and medical information sensed from a user may be received/transmitted from/to the server, via a base station over the air interface 1116. In one embodiment, the transmit/receive element 1122 may be an antenna configured to transmit and/or receive RF signals. In an embodiment, the transmit/receive element 1122 may be an emitter/detector configured to transmit and/or receive IR, UV, or visible light signals, for example. In yet another embodiment, the transmit/receive element 1122 may be configured to transmit and/or receive both RF and light signals. It will be appreciated that the transmit/receive element 1122 may be configured to transmit and/or receive any combination of wireless signals. The transceiver 1120 may be configured to modulate the signals that are to be transmitted by the transmit/receive element 1122 and to demodulate the signals that are received by the transmit/receive element 1122.

The processor 1118 of the device 1100 may be coupled to, and may receive user input data from, the speaker/microphone 1124, the keypad 1126, the display/touchpad 1128 (e.g., a liquid crystal display (LCD) display unit or organic light-emitting diode (OLED) display unit) and/or the peripherals 1138 (e.g., sensors or digital camera). The processor 1118 may also output user data or digital instructions to the speaker/microphone 1124, the keypad 1126, the display/touchpad 1128 and/or the peripherals 1138. In addition, the processor 1118 may access information from, and store data in, any type of suitable memory, such as the non-removable memory 1130 and/or the removable memory 1132. The non-removable memory 1130 may include random-access memory (RAM), read-only memory (ROM), a hard disk, or any other type of memory storage device. The removable memory 1132 may include a subscriber identity module (SIM) card, a memory stick, a secure digital (SD) memory card, and the like. In other embodiments, the processor 218 may access information from, and store data in, memory that is not physically located on the device 1100, such as on a server or a home computer (not shown).

The processor 1118 may receive power from the power source 1134, and may be configured to distribute and/or control the power to the other components in the device 1100. The power source 1134 may be any suitable device for powering the device 1100. For example, the power source 1134 may include one or more dry cell batteries (e.g., nickel-cadmium (NiCd), nickel-zinc (NiZn), nickel metal hydride (NiMH), lithium-ion (Li-ion), etc.), solar cells, fuel cells, and the like.

The processor 1118 may also be coupled to the GPS chipset 1136, which may be configured to provide location information (e.g., longitude and latitude) regarding the current location of the device 1100. In addition to, or in lieu of, the information from the GPS chipset 1136, the device 1100 may receive location information over the air interface 1116 from a base station and/or determine its location based on the timing of the signals being received from two or more nearby base stations. It will be appreciated that the device 1100 may acquire location information by way of any suitable location-determination method while remaining consistent with an embodiment.

The processor 1118 may further be coupled to other peripherals 1138, which may include one or more software and/or hardware modules that provide additional features, functionality and/or wired or wireless connectivity. For example, the peripherals 1138 may include an accelerometer, an e-compass, a satellite transceiver, a digital camera (for photographs and/or video), a universal serial bus (USB) port, a vibration device, a television transceiver, a hands free headset, a Bluetooth® module, a frequency modulated (FM) radio unit, a digital music player, a media player, a video game player module, an Internet browser, a Virtual Reality and/or Augmented Reality (VR/AR) device, an activity tracker, and the like. The peripherals 1138 may include one or more sensors. The sensors may be one or more of a gyroscope, an accelerometer, a hall effect sensor, a magnetometer, an orientation sensor, a proximity sensor, a temperature sensor, a time sensor; a geolocation sensor, an altimeter, a light sensor, a touch sensor, a magnetometer, a barometer, a gesture sensor, a biometric sensor, a humidity sensor and the like.

In some aspects, the present disclosure provides a method of strengthening or in creasing mass of a pelvic floor muscle in a subject. In some embodiments, the method comprises providing, by an electronic device to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising one or more first instructions for the subject to follow. In some embodiments, the electronic device (i) comprises a sensor for sensing adherence by the subject to the first instructions of the one or more first modules, (ii) transmits adherence information, based on the adherence, to a server, and (iii) receives one or more second instructions from the server based on the adherence information. In some embodiments, the method further comprises providing, by the electronic device to the subject, one or more second modules selected from the group consisting of an exercise and/or life style module, a sitz bath module, a pelvic splanchnic nerve and/or pelvic muscle exercise module, and a vagal nerve system and/or gastrointestinal (GI) exercise module, the one or more second modules comprising the one or more second instructions.

In some embodiments, the one or more first modules or the one or more second modules comprise the exercise and/or life style module, and the exercise and/or life style module comprises one or more first instructions to increase a secretion of at least one of growth hormone (GH) and insulin-like growth factor-1 (IGF-1) in the subject. In some embodiments, the exercise and/or life style module comprises one or more first instructions for aerobic, resistance, and/or concurrent exercise. In some embodiments, the instructions for the aerobic, the resistance, and/or the concurrent exercise comprise instructions for one or more independently selected from the group consisting of gripping a finger, tummy time, lifting weights, moving toe, moving ear, and a physical activity. In some embodiments, the physical activity is selected from the group consisting of a structured exercise, kickball, riding a pedal-driven vehicle, jumping, stretching, playing a ball game, skeletal muscle exercise, walking, and aerobic and resistance exercise. In some embodiments, the one or more first instructions comprise instructions for obtaining one or more of deep sleep and adequate sleep.

In some embodiments, the one or more first modules or the one or more second modules comprise the sitz bath module, and the sitz bath module comprises one or more first instructions to maintain non-ischemic status thereby stimulating myosin formation, stimulate rectal ENS maturation, and/or stimulate GLP2 secretion for rectal endocrine cells. In some embodiments, the sitz bath module, and the sitz bath module comprises one or more first instructions to take warm bath, hip bath and/or sitz bath.

In some embodiments, the one or more first modules or the one or more second modules comprise the pelvic splanchnic nerve and/or pelvic muscle exercise module, and the pelvic splanchnic nerve and/or pelvic muscle exercise module comprises one or more first instructions to stimulate rectal ENS maturation, and/or stimulate GLP2 secretion for rectal endocrine cells. In some embodiments, the pelvic splanchnic nerve and/or pelvic muscle exercise module comprises one or more first instructions for cycling away, performing a wheelbarrow, walking, bar hanging, standing upright, trying to touch ceiling, freezing movement, and running. In some embodiments, the one or more first modules or the one or more second modules comprise the vagal nerve system and/or GI exercise module, and the vagal nerve system and/or GI exercise module comprises one or more first instructions to stimulate rectal and/or GI ENS maturation, stimulate GLP2 secretion for rectal and/or GI endocrine cells, and/or decrease inflammation signaling factors. In some embodiments, the one or more first modules or the one or more second modules comprise the vagal nerve system and/or GI exercise module, and the vagal nerve system and/or GI exercise module comprises one or more first instructions to improve anorectal motility. In some embodiments, the one or more first instructions to improve anorectal motility are selected from the group consisting of aerobic exercises including walking, running, cycling, and swimming, walking hand-in-hand with another individual, bar hanging, standing upright, reaching for the ceiling, and limiting motion/playing a freeze game, playing with a small animal, high-intensity playing, adjusting posture, and tapping on a surface. In some embodiments, the vagal nerve system and/or GI exercise module comprises at least one instructions selected from the group consisting of sense stimulation instructions for sight, sound, touch, taste, and smell.

In some embodiments, the vagal nerve stimulation module comprises one or more sense stimulation instructions for sight, and the one or more sense stimulation instructions for sight include one or more instructions to view one or more figures to stimulate autonomic nervous system. In some embodiments, the one or more figures to stimulate the autonomic nervous system are selected from the group consisting of erotic photos, photos of cold, photos of heat, and frightening photos. In some embodiments, the electronic device receives and displays the one or more figures.

In some embodiments, the vagal nerve stimulation module comprises one or more sense stimulation instructions for sound, and the one or more sense stimulation instructions for sound include one or more instructions to hear one or more sounds to cause honor or relaxation. In some embodiments, the one or more instructions to hear one or more sounds to cause horror or relaxation are independently selected from the group consisting of frightening sounds, sounds capable of inducing chill or changes in the skin, raindrop sounds, and sounds capable of inducing sleep. In some embodiments, the electronic device receives and plays the one or more sounds.

In some embodiments, the vagal nerve stimulation module comprises one or more sense stimulation instructions for touch, and the one or more sense stimulation instructions for touch include one or more instructions for abdominal breathing, con trolling rate of breathing, restricting breath for a period of time, controlling heart rate, controlling movement of the GI tract, cold massage, coughing, and skin massage.

In some embodiments, the vagal nerve stimulation module comprises one or more sense stimulation instructions for taste, and the one or more sense stimulation instructions for taste include one or more instructions to eat food to stimulate digestive glands in the subject. In some embodiments, the electronic device receives and display information related to the food.

In some embodiments, the vagal nerve stimulation module comprises one or more sense stimulation instructions for smell, and the one or more sense stimulation instructions for smell include one or more instructions to stimulate digestive glands and/or to relax. In some embodiments, the one or more instructions to stimulate digestive glands and/or to relax stimulate the autonomic nervous system, induce secretion of saliva in the subject, and/or reduce a heart rate of the subject. In some embodiments, the electronic device is configured to release a scent for aroma therapy.

In some embodiments, the subject is a post-operative patient with lower gastrointestinal-operable-congenital anomaly. In some embodiments, the subject has had one or more operations selected from the group consisting of a pull-through surgery, an anorectoplasty, and anoplasty. In some embodiments, prior to or after having the operation, the subject has a condition selected from the group consisting of Hirschsprung disease, an anorectal malformation, and a meningocele. In some embodiments, an age of the subject is 1 month or older, 2 months or older, 3 months or older, 4 months or older, 5 months or older, 6 months or older, 7 months or older, 8 months or older, 9 months or older, 10 months or older, 11 months or older, 12 months or older, 1.5 years or older, 2 years or older, 3 years or older, 4 years or older, or 5 years or older.

In some embodiments, the method is first performed about 1 week after the operation. In some embodiments, the method is performed until the subject is about 20 years old. In some embodiments, the one or more first modules are provided to a guardian of the subject, and the subject received the modules indirectly from the electronic device through the guardian. In some embodiments, the one or more first modules consists of the exercise and/or life style module, the sitz bath module, the pelvic splanchnic nerve and/or pelvic muscle exercise module, and the vagal nerve system and/or gastrointestinal (GI) exercise module.

In some embodiments, the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer. In some embodiments, the sensor comprises a touch sensor, and the subject provides the adherence information to the electronic device using the touch sensor. In some embodiments, the sensor is a detachable peripheral. In some embodiments, the sensor is separate from the display described herein.

In some embodiments, the adherence information includes changes of coordinates on at least two points on the subject over time. In some embodiments, the first instructions comprise changes of coordinates on said points, and the changes of coordinates of the adherence information are compared with the changes of coordinates of the first instructions. In additional embodiments, the coordinates are on at least ten points on the subject over time. In further embodiments, the coordinates are on at least 2, 3, 4, 5, 6, 7, 8, 9, 10, 11, 12, 13, 14, or 15 points on the subject. In further embodiments, the changes of coordinates are within 60, 50, 40, 30, 20, 10, 9, 8, 7, 6, 5, 4, 3, 2, or 1 second(s). In further embodiments, the changes of coordinates are over 0.5, 0.6, 0.7, 0.8, 0.9, or 1 second. In some embodiments, said points are on positions selected from the group consisting of head, shoulder, elbow, wrist, waist, knee, and ankle of the subject.

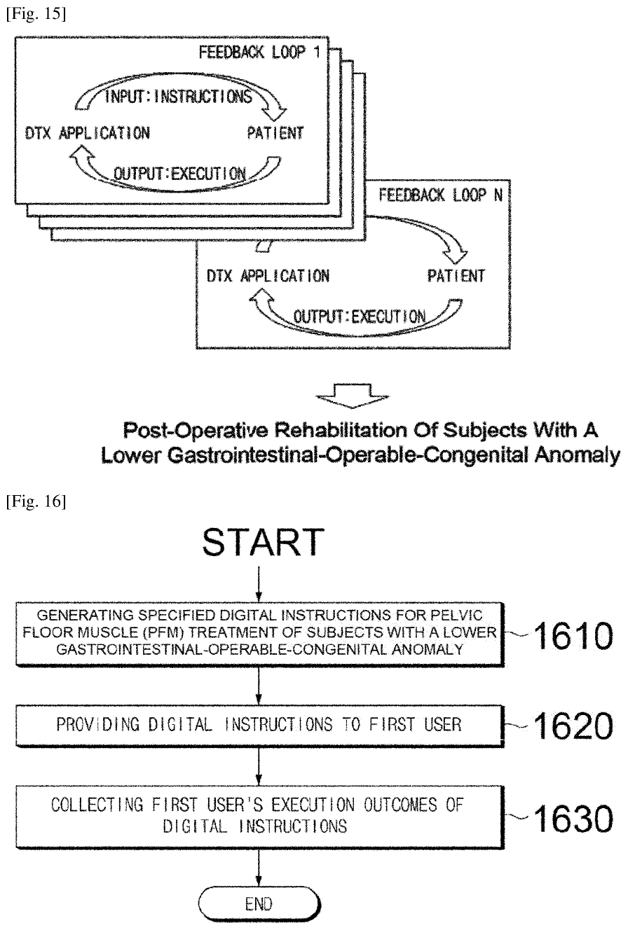

FIG. 15 is a diagram showing a feedback loop for the electronic device and the application for post-operative rehabilitation of subjects with a lower gastrointestinal-operable-congenital anomaly according to one embodiment of the present disclosure. Referring to FIG. 15, the post-operative rehabilitation of subjects with a lower gastrointestinal-operable-congenital anomaly is shown to be achieved by repeatedly executing a single feedback loop several times to regulate the biochemical factors.

Post-operative rehabilitation of subjects with a lower gastrointestinal-operable-congenital anomaly may be more effectively achieved by gradual improvement of an instruction-execution cycle in the feedback loop, compared to the simply repeated instruction-execution cycle during the corresponding course of therapy. For example, the digital instructions and the execution outcomes for the first cycle are given as input values and output values in a single loop, but new digital instructions may be generated by reflecting input values and output values generated in this loop using a feedback process of the loop to adjust the input for the next loop when the feedback loop is executed N times. This feedback loop may be repeated to deduce patient-customized digital instructions and maximize a therapeutic effect at the same time.

As such, in the electronic device and the application for post-operative rehabilitation of subjects with a lower gastrointestinal-operable-congenital anomaly according to one embodiment of the present disclosure, the patient's digital instructions provided in the previous cycle (for example, a N−1^{st} cycle), and the data on instruction execution outcomes may be used to calculate the patient's digital instructions and execution outcomes in this cycle (for example, a N^{th} cycle). That is, the digital instructions in the next loop may be generated based on the patient's digital instructions and execution outcomes of the digital instructions calculated in the previous loop. In this case, various algorithms and statistical models may be used for the feedback process, when necessary. As described above, in the electronic device and the application for post operative rehabilitation of subjects with a lower gastrointestinal-operable-congenital anomaly according to one embodiment of the present disclosure, it is possible to optimize the patient-customized digital instructions suitable for the patient through the rapid feedback loop.

FIG. 16 is a flowchart illustrating operations in the digital application for post operative rehabilitation of subjects with a lower gastrointestinal-operable-congenital anomaly according to one embodiment of the present disclosure. Referring to FIG. 16, the digital application for post-operative rehabilitation of subjects with a lower gastrointestinal-operable-congenital anomaly according to one embodiment of the present disclosure may first generate specified digital instructions based on the one or more instructions (1610). 1610 may generate a one or more instructions by applying imaginary parameters about the patient's environments, behaviors, emotions, and cognition to the mechanism of action in and the therapeutic hypothesis for a lower gastrointestinal-operable-congenital anomaly. In this case, in 1610, the one or more instructions may be generated based on the biochemical factors (for example, growth hormone (GH) and insulin-like growth factor-1 (IGF-1)) for post-operative rehabilitation of subjects with a lower gastrointestinal-operable-congenital anomaly. Meanwhile, in 1610, the one or more instructions may be generated based on the inputs from the healthcare provider or expert reviewer. In this case, a one or more instructions may be generated based on the information collected by the doctor when diagnosing a patient, and the prescription outcomes recorded based on the information. Also, in 1610, the one or more instructions may be generated based on the information (for example, basal factors, medical information, digital therapeutics literacy, etc.) received from the patient.

Then, the digital instructions may be provided to a patient (1620). In this case, the digital instructions may be provided in the form of digital instructions which are associated with behaviors and in which the patient's instruction adherence may be monitored using a sensor, or provided in the form of digital instructions in which a patient is allowed to directly input the execution outcomes. Generally, the one or more instructions can be independently selected from the group consisting of an alarm, a silent alarm or a vibration, an instruction to proceed, or an instruction to stop.

After the patient executes the presented digital instructions, the patient's execution outcomes of the digital instructions may be collected (1630). In 1630, the execution outcomes of the digital instructions may be collected by monitoring the patient's adherence to the digital instructions as described above, or allowing the patient to input the execution outcomes of the digital instructions.

Meanwhile, the digital application for post-operative rehabilitation of subjects with a lower gastrointestinal-operable-congenital anomaly according to one embodiment of the present disclosure may repeatedly execute operations several times, wherein the operations include generating the digital instruction and collecting the patient's execution outcomes of the digital instructions. In this case, the generating of the digital instruction may include generating the patient's digital instructions for this cycle based on the patient's digital instructions provided in the previous cycle and the execution outcome data on the patient's collected digital instructions provided in the previous cycle.

As described above, according to the digital application for post-operative rehabilitation of subjects with a lower gastrointestinal-operable-congenital anomaly according to one embodiment of the present disclosure, the reliability of the post operative rehabilitation of subjects with a lower gastrointestinal-operable-congenital anomaly may be ensured by deducing the mechanism of action in and the therapeutic hypothesis for the lower gastrointestinal-operable-congenital anomaly in consideration of the biochemical factors for lower gastrointestinal-operable-congenital anomaly, presenting the digital instructions to a patient based on the mechanism of action in and the therapeutic hypothesis for lower gastrointestinal-operable-congenital anomaly, and collecting and analyzing the outcomes of the digital instructions.

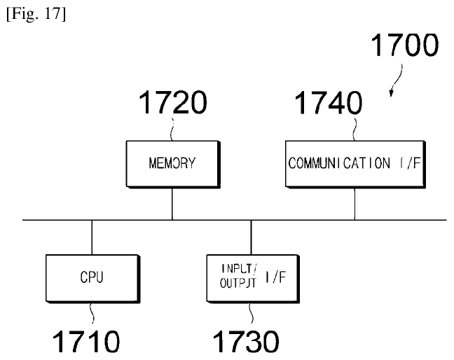

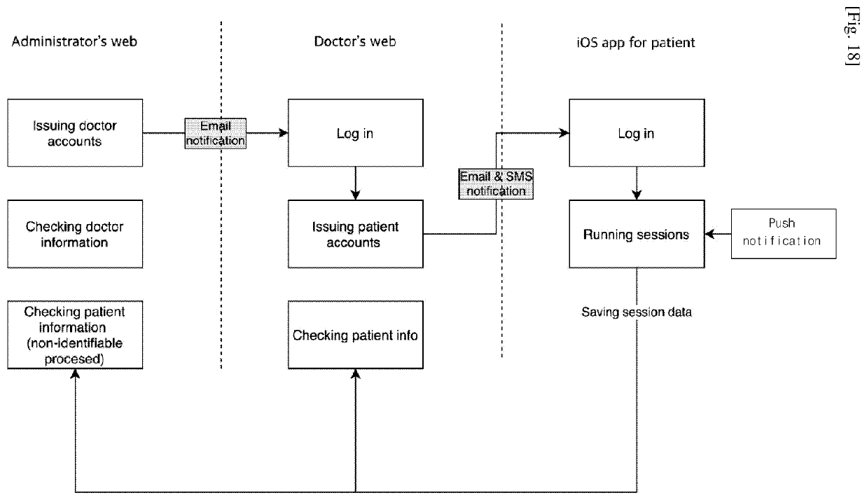

FIG. 17 is a diagram showing a hardware configuration of the electronic device for post-operative rehabilitation of subjects with a lower gastrointestinal-operable-congenital anomaly according to one embodiment of the present disclosure.

Referring to FIG. 17, hardware 1700 of the electronic device for post-operative rehabilitation of subjects with a lower gastrointestinal-operable-congenital anomaly according to one embodiment of the present disclosure may include a CPU 1710, a memory 1720, an input/output 1/F 1730, and a communication I/F 1740.

The CPU 1710 may be a processor configured to execute a digital application for post-operative rehabilitation of subjects with a lower gastrointestinal-operable-congenital anomaly stored in the memory 1720, process various data for post operative rehabilitation of subjects with a lower gastrointestinal-operable-congenital anomaly and execute functions associated with the post-operative rehabilitation of subjects with a lower gastrointestinal-operable-congenital anomaly. That is, the CPU 1710 may act to execute functions by executing the digital application for post operative rehabilitation of subjects with a lower gastrointestinal-operable-congenital anomaly stored in the memory 1720.

The memory 1720 may have a digital application for post-operative rehabilitation of subjects with a lower gastrointestinal-operable-congenital anomaly stored therein. Also, the memory 1720 may include the data used for the digital post-operative rehabilitation of subjects with a lower gastrointestinal-operable-congenital anomaly included in the database, for example, the patient's digital instructions and instruction execution outcomes, the patient's medical information, and the like.

A plurality of such memories 1720 may be provided, when necessary. The memory 1720 may be a volatile memory or a non-volatile memory. When the memory 1720 is a volatile memory, RAM, DRAM, SRAM, and the like may be used as the memory 1720. When the memory 1720 is a non-volatile memory, ROM, PROM, EAROM, EPROM, EEPROM, a flash memory, and the like may be used as the memory 1720. Examples of the memories 1720 as listed above are given by way of illustration only, and are not intended to limit the present disclosure.

The input/output 1/F 1730 may provide an interface in which input apparatuses (not shown) such as a keyboard, a mouse, a touch panel, and the like, and output apparatuses such as a display (not shown), and the like may transmit and receive data (e.g., wirelessly or by hardline) to the CPU 1710.

The communication I/F 1740 is configured to transmit and receive various types of data to/from a server, and may be one of various apparatuses capable of supporting wire or wireless communication. For example, the types of data on the aforementioned digital behavior-based therapy may be received from a separately available external server through the communication I/F 1740.

According to the electronic device and the application for post-operative rehabilitation of subjects with a lower gastrointestinal-operable-congenital anomaly according to the present disclosure, a reliable electronic device and application capable of post-operative rehabilitation of subjects with a lower gastrointestinal-operable-congenital anomaly may be provided by deducing a mechanism of action for the underlying condition in the subject, in consideration of biochemical factors for the underlying condition, presenting digital instructions to a patient, and collecting and analyzing execution outcomes of the digital instructions.

FIG. 18 depicts a flow chart illustrating a system for post-operative rehabilitation of subjects with a lower gastrointestinal-operable-congenital anomaly, the system comprising an administrative portal (e.g., Administrator's web), a healthcare provider portal (e.g., Doctor's web) and a digital apparatus configured to execute a digital application (e.g., an application or ‘app’) for post-operative rehabilitation of subjects with a lower gastrointestinal-operable-congenital anomaly. Among other things, the Administrator's portal allows an administrator to issue doctor accounts, review doctor in formation, and review de-identified patient information. Among other things, the Healthcare Provider's portal allows a healthcare provider (e.g., a doctor) to issue patient accounts, and review patient information (e.g., age, prescription information, and status for having completed one or more post-operative rehabilitation sessions). Among other things, the digital application allows a patient access to complete one or more post-operative rehabilitation sessions.

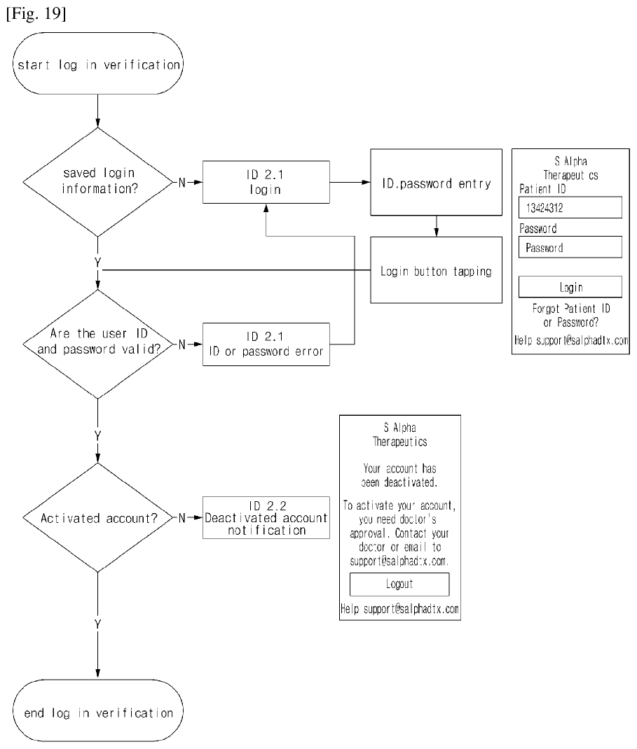

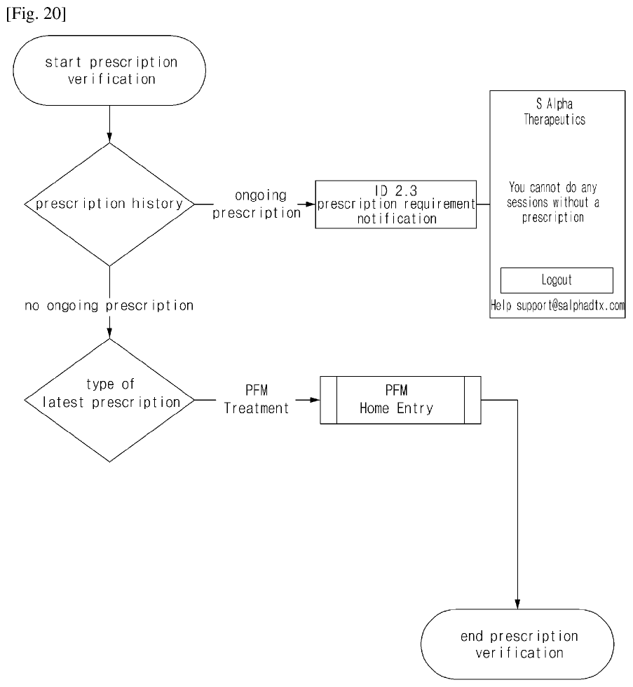

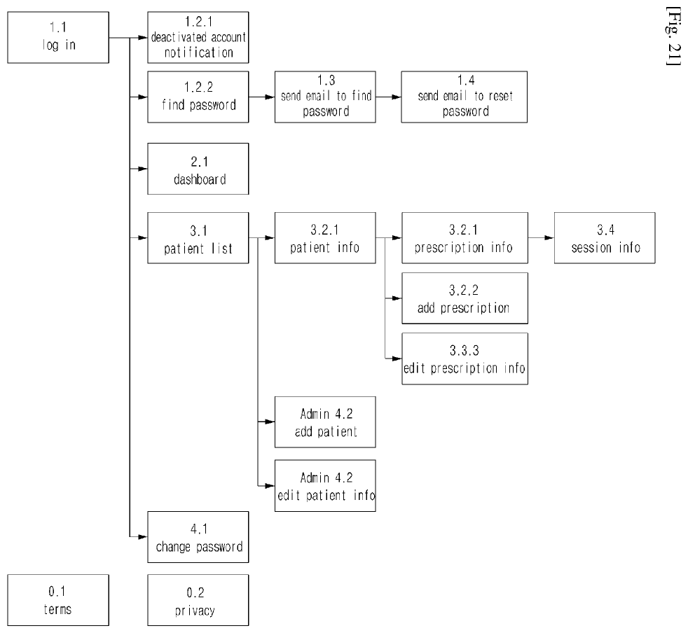

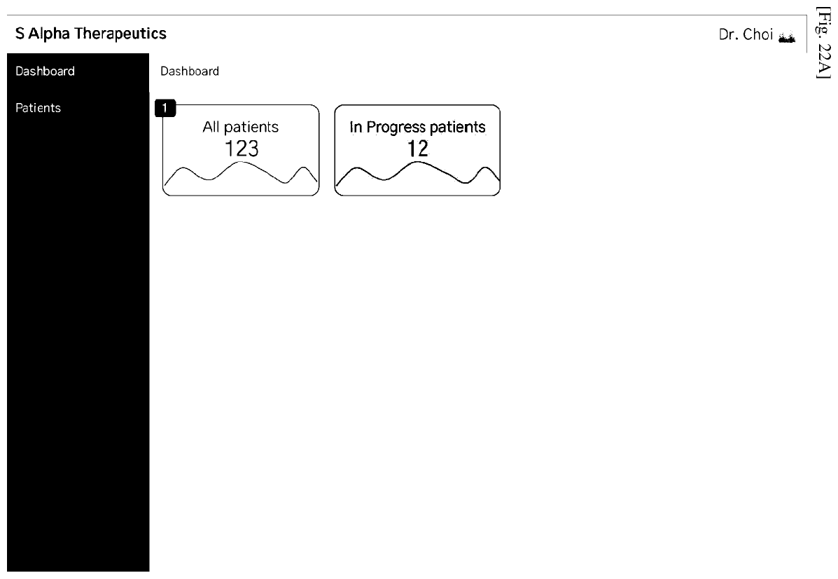

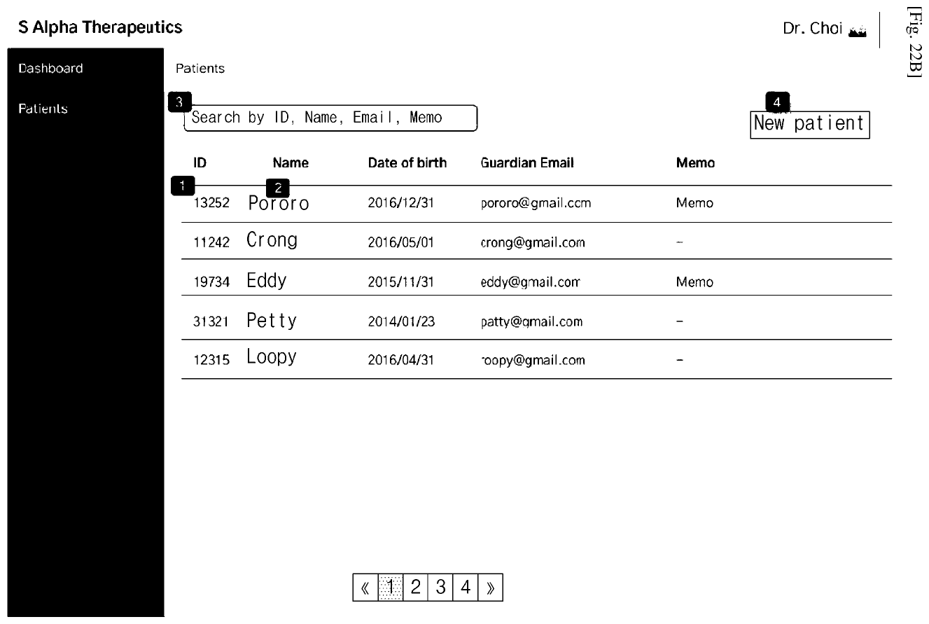

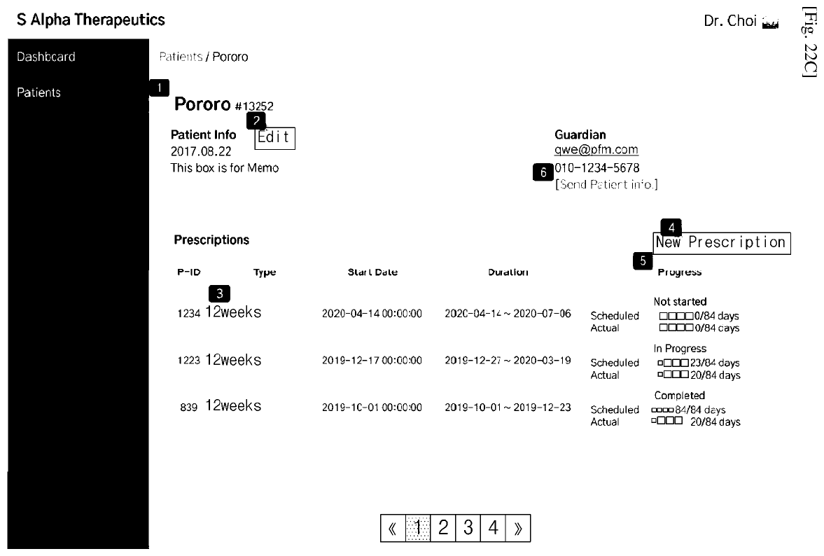

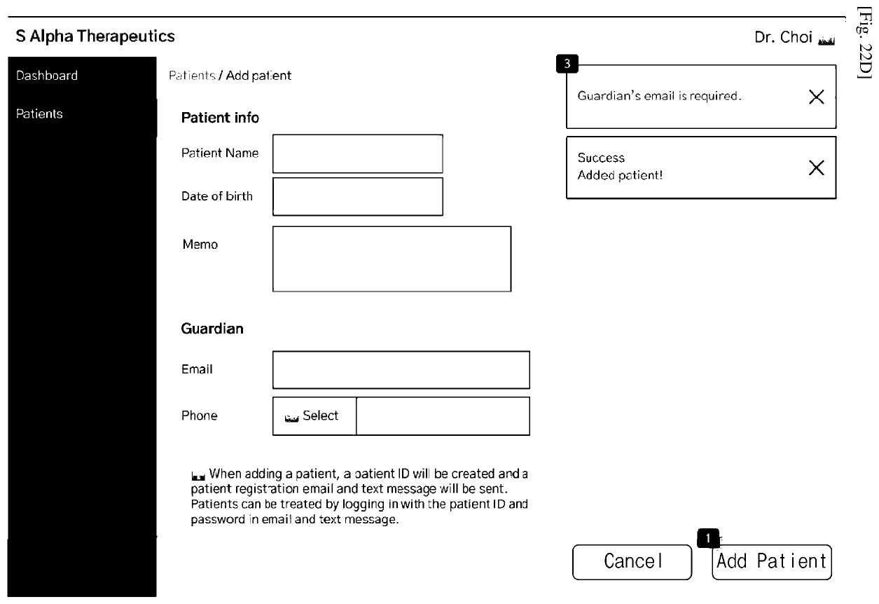

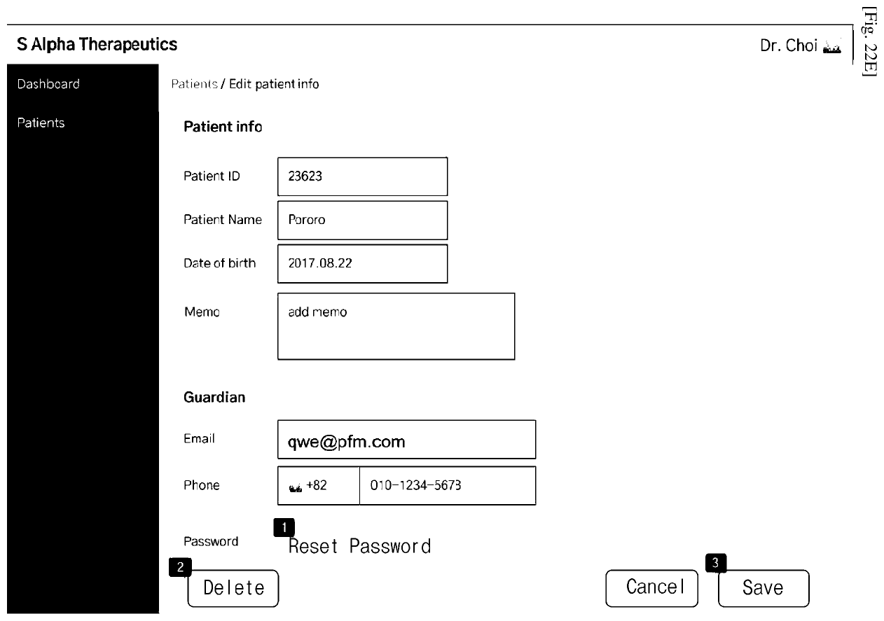

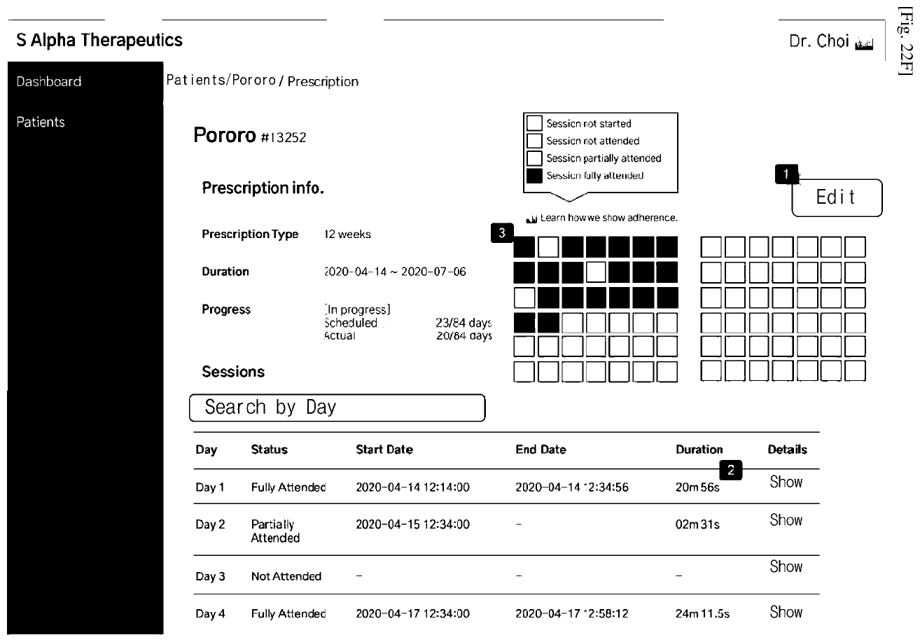

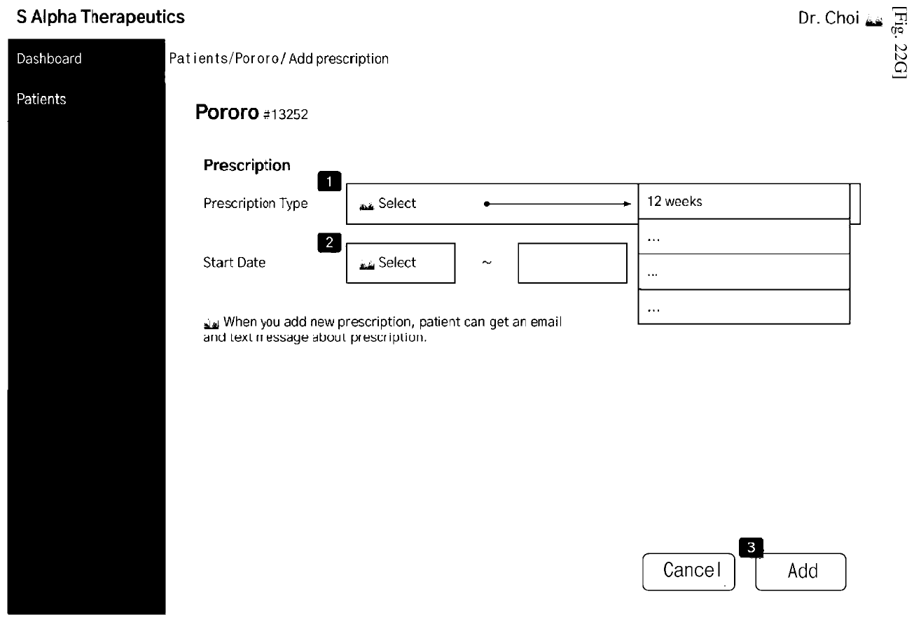

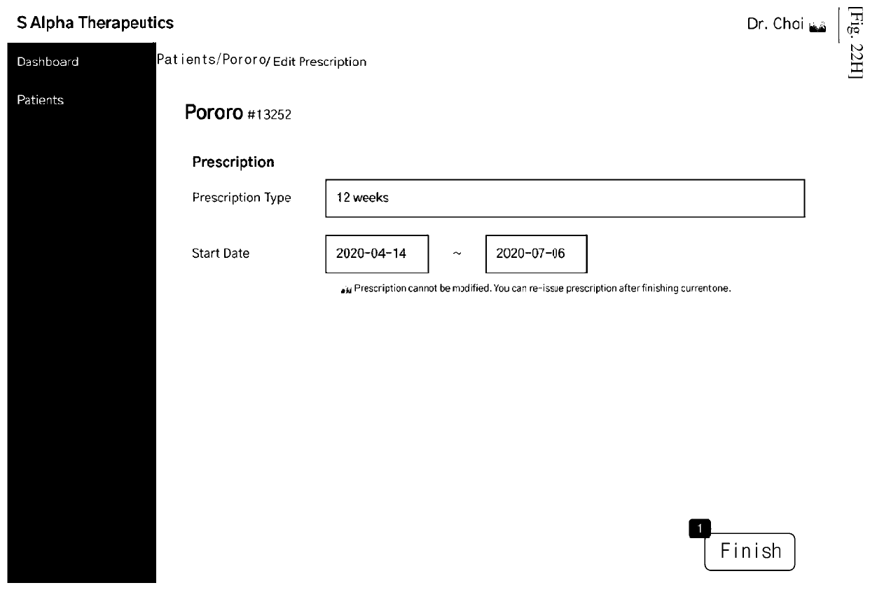

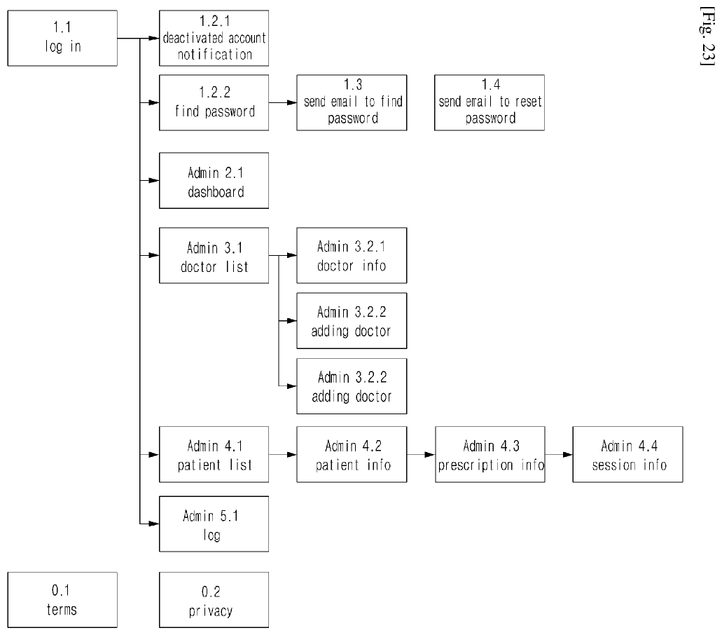

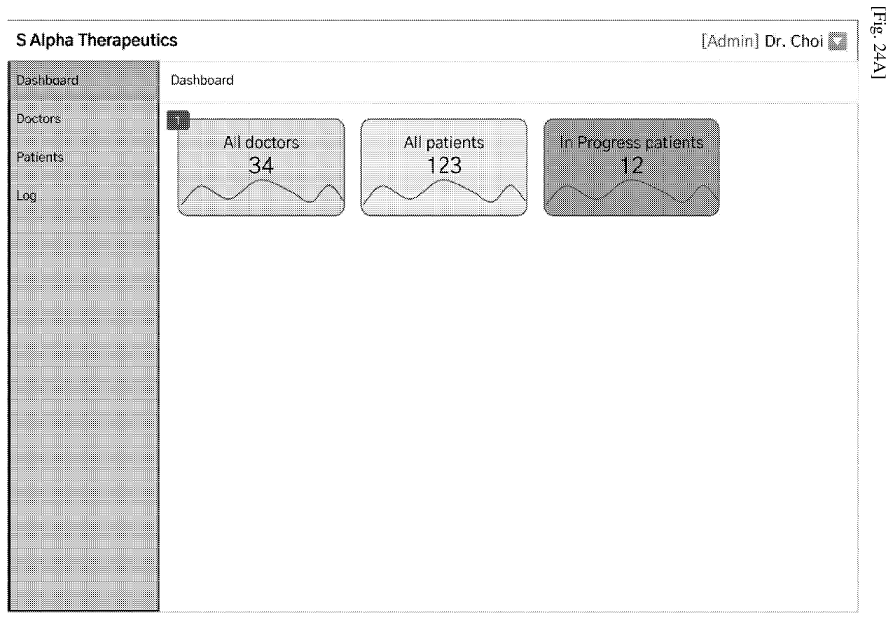

FIG. 19 depicts a flow chart illustrating an execution flow for login verification during a splash process at the starting of the digital application. Similarly, FIG. 20 depicts a flow chart illustrating an execution flow for prescription verification during a splash process at the starting of the digital application. The prescription verification process may comprise, for example, determining if the treatment period has expired, or determining if, based on the prescription, the subject's sessions for the day have been completed (e.g., the subject is compliant with the prescription). In such instances, the digital apparatus may notify the subject that there are no post-operative rehabilitation sessions available to be completed.

In some embodiments, the healthcare provider portal provides a healthcare provider with one or more options, and the one or more options provided to the healthcare provider are selected from the group consisting of adding or removing the subject, viewing or editing personal information for the subject, viewing adherence information for the subject, viewing a result of the subject for one or more at least partially completed post-operative rehabilitation sessions, prescribing one or more post operative rehabilitation sessions to the subject, altering a prescription for one or more post-operative rehabilitation sessions, and communicating with the subject. In some embodiments, the one or more options comprise the viewing or editing personal in formation for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed post-operative rehabilitation sessions to be performed by the subject, and a number of scheduled or prescribed post operative rehabilitation sessions to be performed by the subject per day. In some embodiments, the one or more options comprise the viewing the adherence information, and the adherence information of the subject comprises one or more of a number of scheduled or prescribed post-operative rehabilitation sessions completed by the subject, and a calendar identifying one or more days on which the subject completed, partially completed, or did not complete one or more scheduled or prescribed post operative rehabilitation sessions. In some embodiments, the one or more options comprise the viewing the result of the subject, and the result of the subject for one or more at least partially completed post-operative rehabilitation sessions comprises one or more selected from the group consisting of a time at which the subject started a scheduled or prescribed post-operative rehabilitation session, a time at which the subject ended a scheduled or prescribed post-operative rehabilitation session, and an indicator of whether the scheduled or prescribed post-operative rehabilitation session was fully or partially completed.

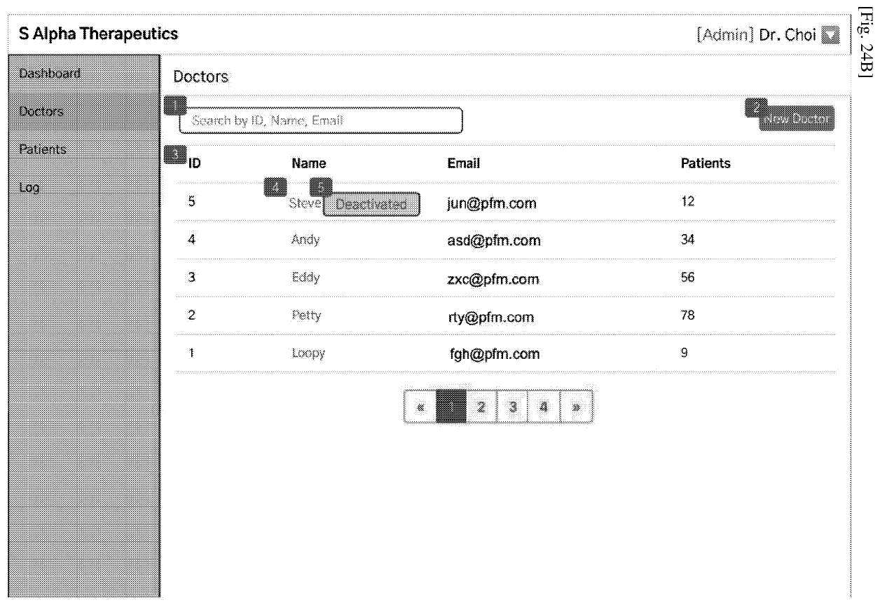

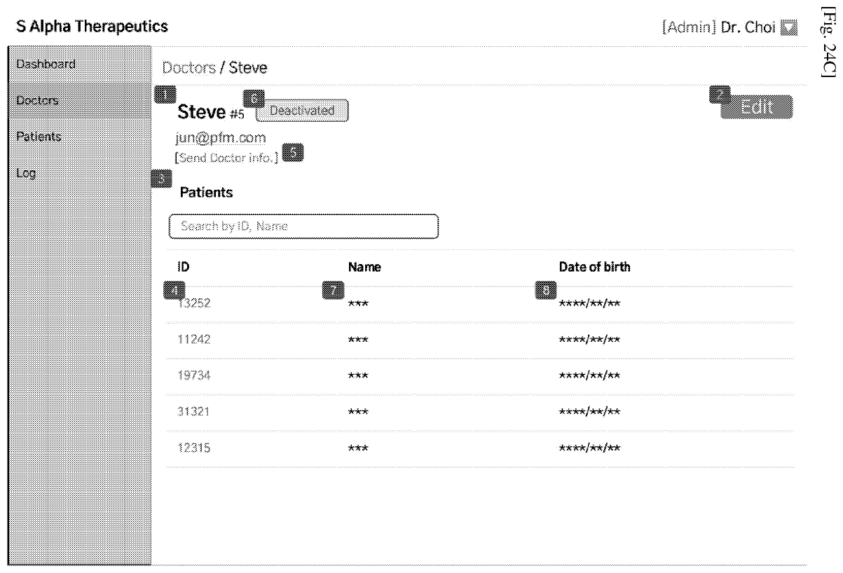

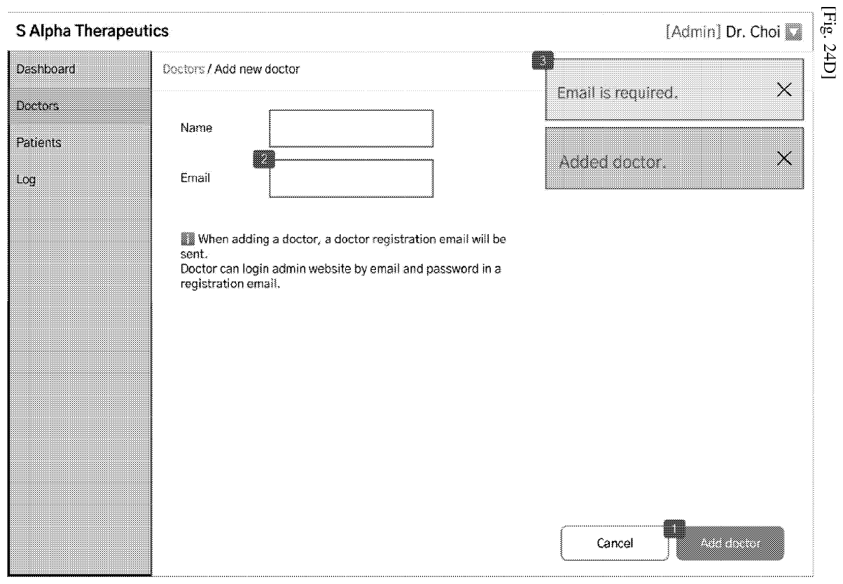

FIG. 22A depicts a dashboard of a healthcare provider portal. (1) The number of all patients associated with the present doctor's account. A graph may be used to show the number of patients who have opened the digital application for patient per day in the most recent 90 days. The number of patients in progress may also be viewed. A graph may be used to show the number of patients who have completed the sessions per day in the most recent 90 days. FIG. 22B depicts a patient tab in a healthcare provider portal, the patient tab displaying a list of patients. As shown, (1) Patient ID (the unique identification number temporarily given to each patient when adding them on the list), (2) Patient Name, (3) Search bar for searching by ID, Name, Email, Memo, etc., and (4) Add New Patient button for adding new patients. FIG. 22C depicts a patient tab in a healthcare provider portal, the patient tab displaying detailed information on a given patient. As shown, (1) detailed patient information, (2) a button for editing patient in formation, (3) prescription information, (4) a button for adding a new prescription, (5) displays a progress status for different each prescription, and (6) a button or link for sending an email to the patient. FIG. 22D depicts a patient tab in a healthcare provider portal for adding a new patient. As shown, (1) shows a button for adding a new patient, and (3) shows an error message displayed when required patient information has not been provided. FIG. 22E depicts a patient tab in a healthcare provider portal for editing information of an existing patient. As shown, (1) is a button or link for resetting a password, (2) is a button for deleting a given patient, and (3) is a button for saving changes. FIG. 22F depicts a patient tab in a healthcare provider portal that displays detailed prescription information for a given patient. As shown, (1) is a button for editing prescription information, (2) displays the duration of the sessions attended by the patient or subject, and (3) shows an overview the treatment progress. Seven days are represented as a line or row of 7 squares. For 12 weeks, each 6 weeks may be presented separately. Different colors may be used to discern session statuses (e.g., grey for sessions not started, red for sessions not attended, yellow for sessions partially attended, and green for sessions fully attended). FIGS. 22G-H depict a patient tab in a healthcare provider portal for editing prescription information for a given patient.

In some embodiments, the administrative portal provides an administrator with one or more options, and the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed post-operative rehabilitation sessions, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed post-operative rehabilitation sessions completed by the subject, and a calendar identifying one or more days on which the subject completed, partially completed, or did not complete one or more scheduled or prescribed post operative rehabilitation sessions. In some embodiments, the one or more options comprise the viewing the result of the subject, and the result of the subject for one or more at least partially completed post-operative rehabilitation sessions comprises one or more selected from the group consisting of a time at which the subject started a scheduled or prescribed post-operative rehabilitation session, a time at which the subject ended a scheduled or prescribed post-operative rehabilitation session, and an indicator of whether the scheduled or prescribed post-operative rehabilitation session was fully or partially completed.

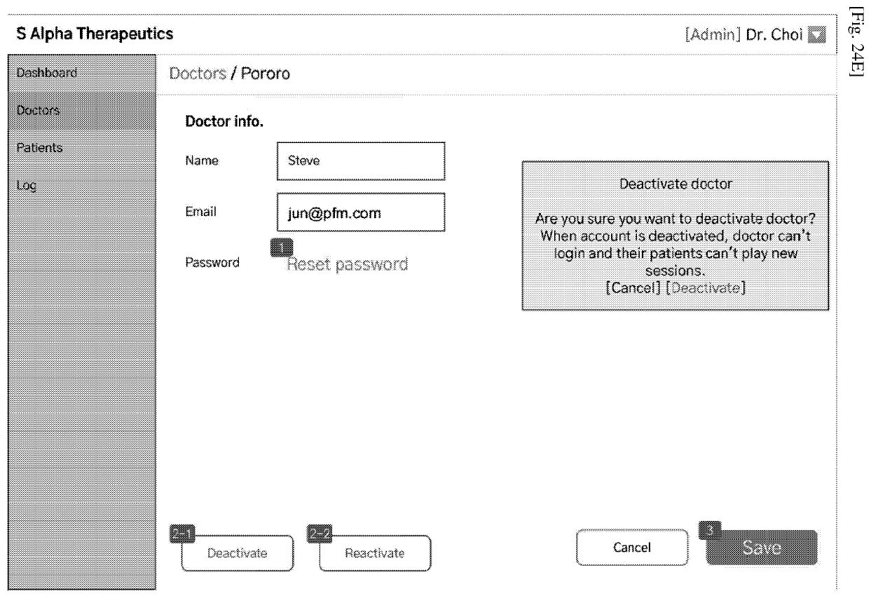

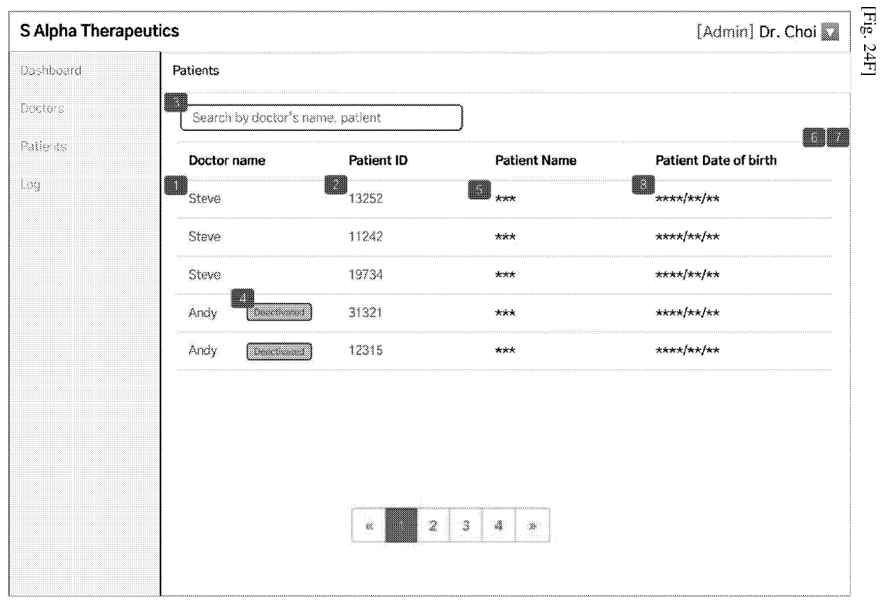

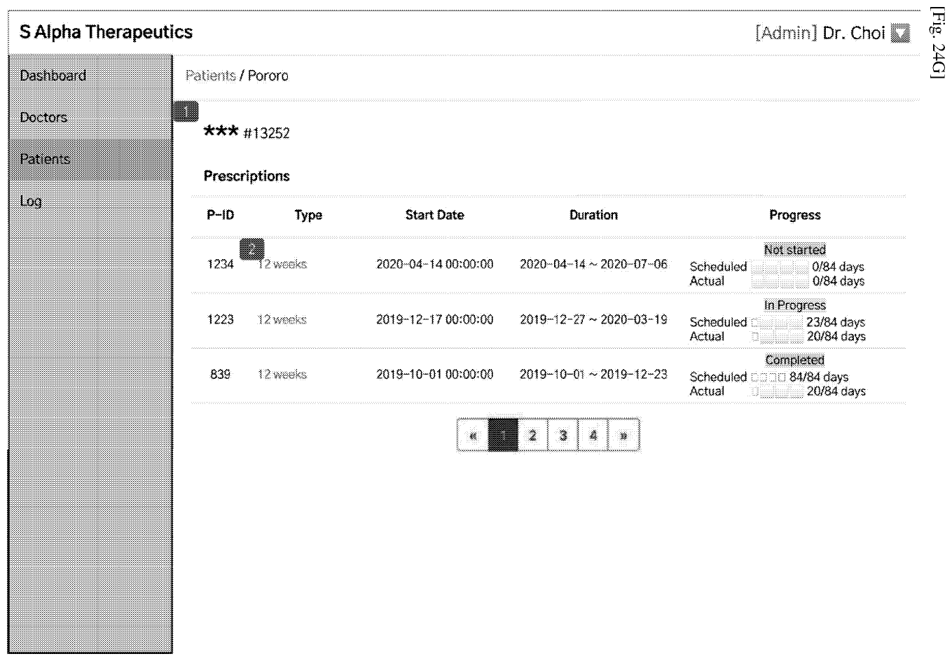

FIG. 24A depicts (A) a dashboard of an administrative portal. As shown, (1) shows the number of doctors. A graph may be used to show the number of doctors that have visited the digital application per day in the most recent 90 days, (2) The number of all patients associated with the any doctor's account. A graph may be used to show the number of patients who have opened the digital application for patient per day in the most recent 90 days. The number of patients in progress may also be viewed. A graph may be used to show the number of patients who have completed the sessions per day in the most recent 90 days. FIG. 24B depicts a doctor tab in an administrative portal, the doctor tab displaying a list of doctors. As shown, (1) is a search bar for searching for various doctors by name, email, etc., (2) shows a button for adding a new doctor, (3) is the doctor's ID, (4) is a button for viewing detailed doctor information, and (5) shows deactivated doctor accounts. FIG. 24C depicts a doctor tab in an administrative portal, the doctor tab displaying a list of patients being cared for by a given doctor, with patient-identifying information redacted (*). As shown, (1) is the doctor's account information, (2) is a button for editing the doctor's account information, (3) is a list of patients being cared for by the doctor, (4) is a list of patient ID numbers, (5) a link or button for sending the doctor a registration email, (6) a notification that the doctor's account has been deactivated, which only appears for deactivated accounts, and (7 and 8) redacted or de-identified patient information. FIG. 24D depicts a doctor tab in an administrative portal for adding a new doctor. FIG. 24E depicts a doctor tab in an administrative portal for editing information of an existing doctor, including activating or de activating a doctor's account. FIG. 24F depicts a patient tab in an administrative portal that displays information for one or more patients, wherein sensitive information is redacted. FIG. 24G depicts a patient tab in an administrative portal that displays detailed patient or prescription information for a given patient. FIG. 24H depicts a patient tab in an administrative portal that displays detailed prescription information for a given patient. FIG. 25 provides a table showing privileges for the doctors using the healthcare provider portal and the administrators using the administrative portal.

In some aspects, the present disclosure provides a computing system for strengthening or increasing mass of a pelvic floor muscle in a subject in need thereof. In some embodiments, the computing system comprises a display configured to provide, to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising one or more first instructions for the subject to follow. In some embodiments, the computing system comprises a sensor configured to sense adherence by the subject to the instructions of the one or more first modules. In some embodiments, the computing system comprises a transmitter configured to transmit adherence information, based on the adherence, to a server. In some embodiments, the computing system comprises a receiver configured to receive, from the server, one or more second instructions based on the adherence in formation.

Any of the computing systems mentioned herein can utilize any suitable number of subsystems. In some embodiments, a computing system includes a single computer apparatus, where the subsystems can be the components of the computer apparatus. In other embodiments, a computing system can include multiple computer apparatuses, each being a subsystem, with internal components. A computing system can include desktop and laptop computers, tablets, mobile phones and other mobile devices.

The subsystems can be interconnected via a system bus. Additional subsystems include a printer, keyboard, storage device(s), and monitor, which is coupled to display adapter. Peripherals and input/output (I/O) devices, which couple to I/O controller, can be connected to the computing system by any number of connections known in the art such as an input/output (I/O) port (e.g., USB, FireWire®. For example, an I/O port or external interface (e.g., Ethernet, Wi-Fi, etc.) can be used to connect computing system to a wide area network such as the Internet, a mouse input device, or a scanner. The interconnection via system bus allows the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) can embody a computer readable medium. Another subsystem is a data collection device, such as a camera, microphone, accelerometer, and the like. Any of the data mentioned herein can be output from one component to another component and can be output to the user.

A computing system can include a plurality of the same components or subsystems, e.g., connected together by external interface or by an internal interface. In some embodiments, computing systems, subsystem, or apparatuses can communicate over a network. In such instances, one computer can be considered a client and another computer a server, where each can be part of a same computing system. A client and a server can each include multiple systems, subsystems, or components.

Aspects of embodiments can be implemented in the form of control logic using hardware (e.g., an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments described herein using hardware and a combination of hardware and software.

In some aspects, the present disclosure provides a non-transitory computer readable medium having stored thereon software instructions for strengthening or increasing mass of a pelvic floor muscle in a subject in need thereof that, when executed by a processor, cause the processor to display, by an electronic device to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising instructions for the subject to follow. In some aspects, the present disclosure provides a non-transitory computer readable medium having stored thereon software instructions for strengthening or increasing mass of a pelvic floor muscle in a subject in need thereof that, when executed by a processor, cause the processor to sense, by a sensor in the electronic device, adherence by the subject to the instructions of the one or more first modules. In some aspects, the present disclosure provides a non-transitory computer readable medium having stored thereon software instructions for strengthening or increasing mass of a pelvic floor muscle in a subject in need thereof that, when executed by a processor, cause the processor to transmit, by the electronic device, adherence information, based on the adherence, to a server. In some aspects, the present disclosure provides a non-transitory computer readable medium having stored thereon software instructions for strengthening or increasing mass of a pelvic floor muscle in a subject in need thereof that, when executed by a processor, cause the processor to receive, from the server, one or more second instructions based on the adherence information.

Any of the software components or functions described in this application can be implemented as software code to be executed by a processor using any suitable computer language such as, for example, Java, C, C++,C #, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium can be any combination of such storage or transmission devices.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computing system), and can be present on or within different computer products within a system or network. A computing system can include a monitor, printer, or other suitable display for providing any of the results mentioned herein to a user.

Any of the methods described herein can be totally or partially performed with a computer system including one or more processors, which can be configured to perform the steps. Thus, embodiments can be directed to computer systems configured to perform the steps of any of the methods described herein, with different components performing a respective steps or a respective group of steps. Although presented as numbered steps, steps of methods herein can be performed at a same time or in a different order. Additionally, portions of these steps can be used with portions of other steps from other methods. Also, all or portions of a step can be optional. Additionally, any of the steps of any of the methods can be performed with modules, units, circuits, or other approaches for performing these steps.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs). A processor in association with software may be used to implement a radio frequency transceiver for use in a WTRU, UE, terminal, base station, RNC, or any host computer.

### Example

An application is prepared to include patient application log-in, daily activity, and checklist. At the patient applicant log-in process, patient IDs were automatically issued when doctors register patients, and the doctors input patient information, such as name, E-mail address, phone number and date of birth. As daily activities, instructions for voluntary activities are provided to perform a one-minute action followed by a one-minute break (drinking water) 5 times a day. The action includes standing posture, raising arms and heels, squatting, and bending the legs forward, all of which are sensed by a motion sensor or a camera. The instructions include 12 weeks prescription, 10 minutes daily activities (including rest). For the daily action, when the voluntary activity is performed, the application checks whether it is performed by using the sensor. In addition, as checklists, inductions of action are provided with respect to sleep, skin massage and sitz bath. The sleeping instructions include guidance to fall asleep before 10:00 (every day), the skin massage instructions include checking whether the body, arm, and leg are massaged (daily), and the sitz bath instructions include checking whether the sitz bath is performed through guide and timer and checking whether defecation is present (e.g., twice a week). For the checklist, when the subject proceeds according to the guide, the subject is asked to check whether the induction of action is performed. Once the instructions are followed, the application offers rewards for farm decoration. A doctor monitors the adherence of the instructions via web.

The voluntary action includes sensing the posture of attention, raising arms and heels, squatting, and bending forward with legs, make the character on the screen move. FIG. 25 depicts an exemplary interfaces for the start screen, voluntary activity, induction of action, and provision of rewards. The instructions include repeating 1 minute of voluntary action and 1 minute of rest 5 times a day (10 minutes a day). The induction of action includes inducing sleep (daily), skin massage (daily), and sitz bath (twice a week), and the instructions include checking whether it is executed. Daily farm decoration rewards are provided by checking whether voluntary actions, sleep, skin massage, and sitz bathing are performed.

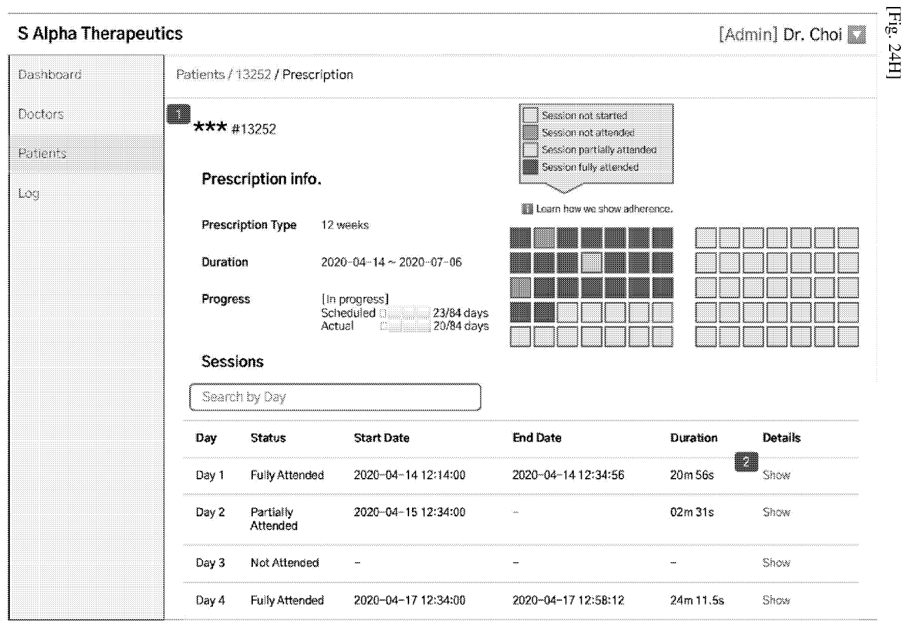

For the voluntary activates, four voluntary activities are prescribed, followed by one activity per day for 12 weeks. The four voluntary activities consist of a story about planting crops, avoiding obstacles, catching moles, and chasing wild. After the prescription, planting crops on the 1st day, avoiding obstacles on the 2nd day, catching a mole on the 3rd day, and chasing a wild boar on the 4th day are proceeded, and then planting crops begins again from the 5th day. Each of the four voluntary activities senses the following postures to make the character move: planting crops (Day 1, Day 5 . . . ) by raising arms and heels, and squatting; avoiding Obstacles (Day 2, Day 6 . . . ) by raising arms and heels, stretching legs forward, and bending; mole Catching (Day 3, Day 7 . . . ) by attention and squatting; and wild boar chase (Day 4, Day 8 . . . ) for attention posture, raising arms and heels, squatting, stretching legs forward, and bending. Exemplary game list is shown in FIG. 26.

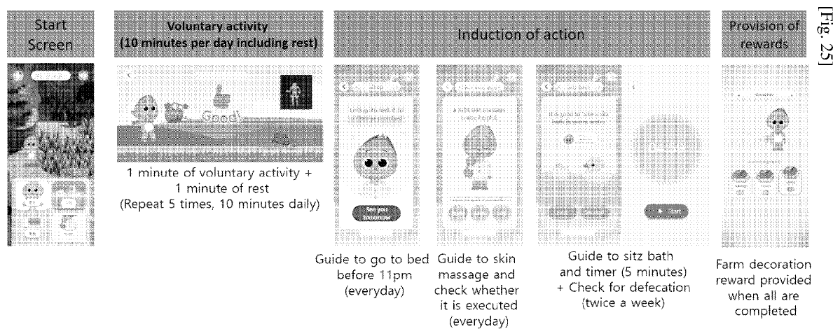

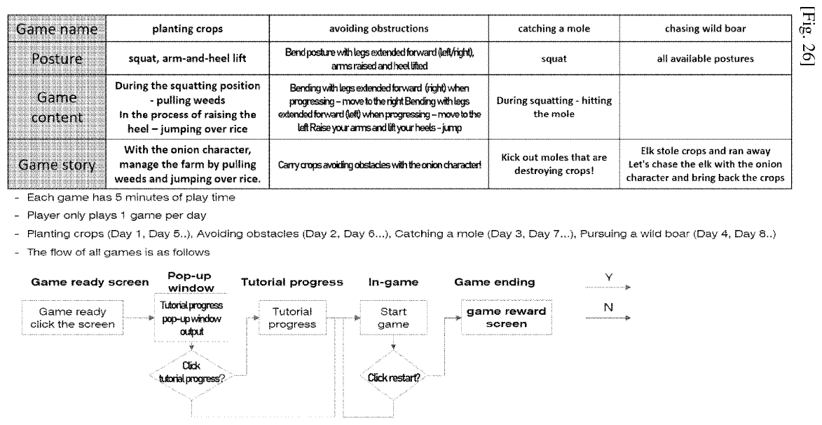

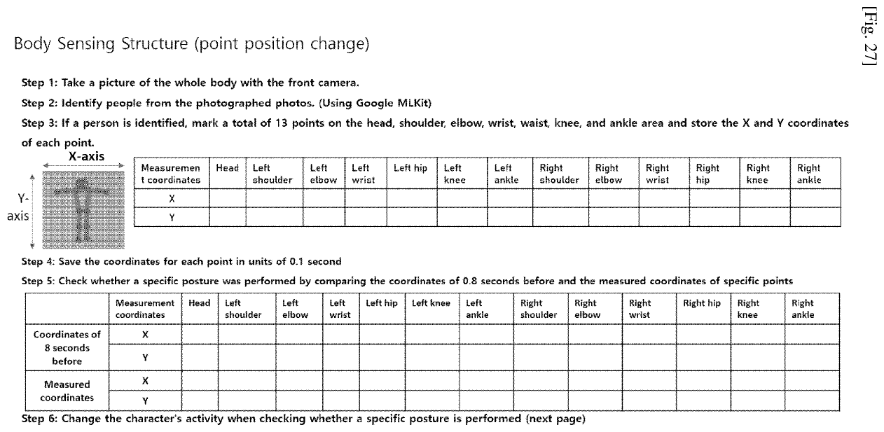

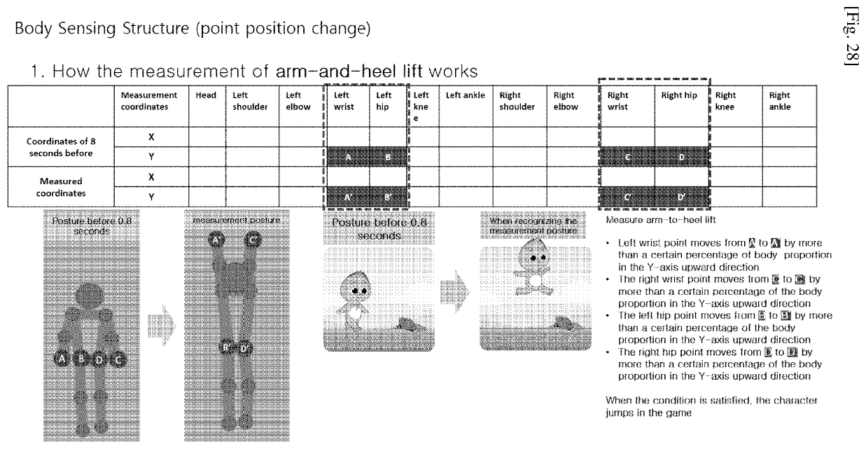

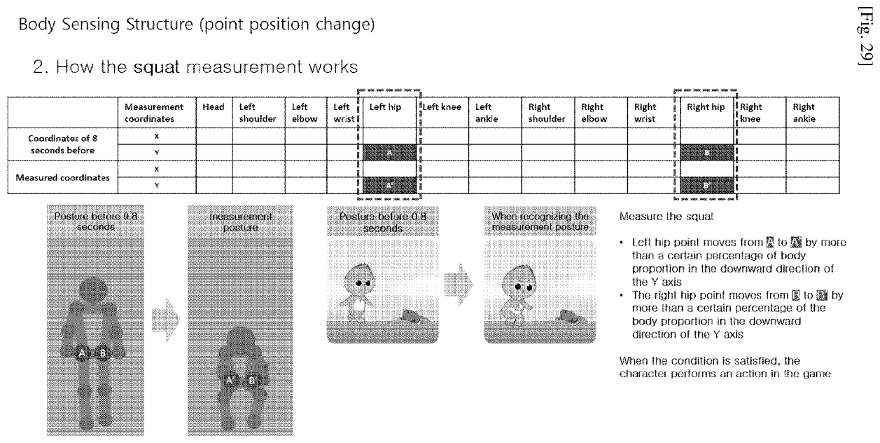

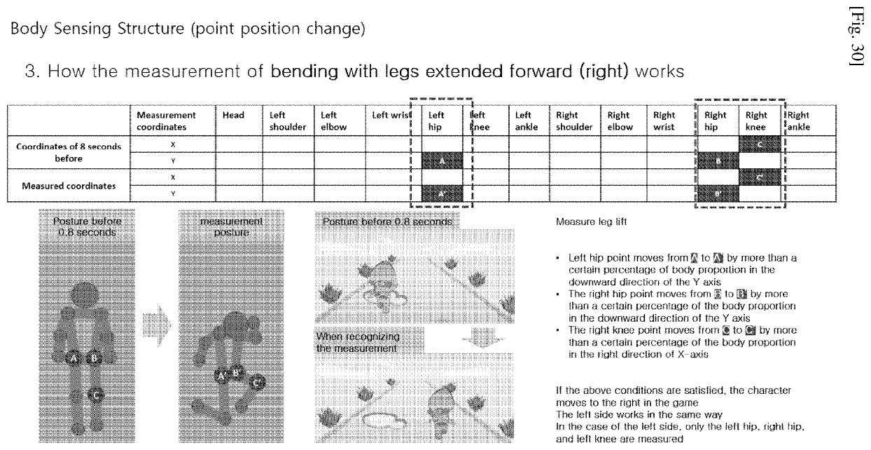

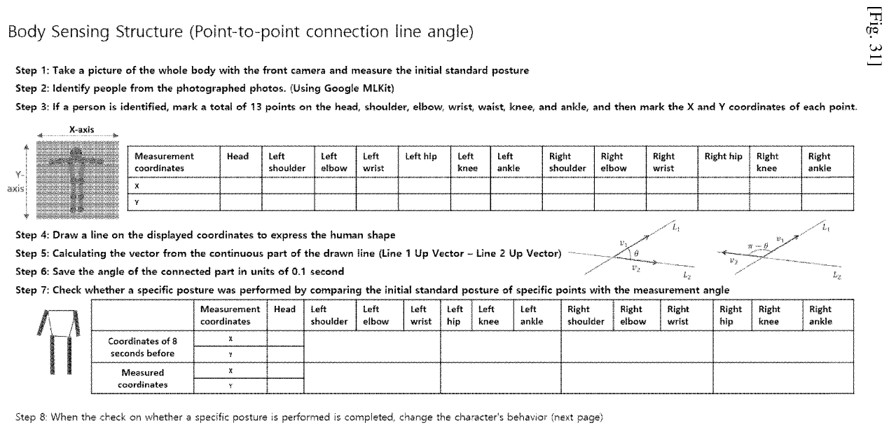

The exemplary body sensing during the activities is depicted in FIGS. 27-32.

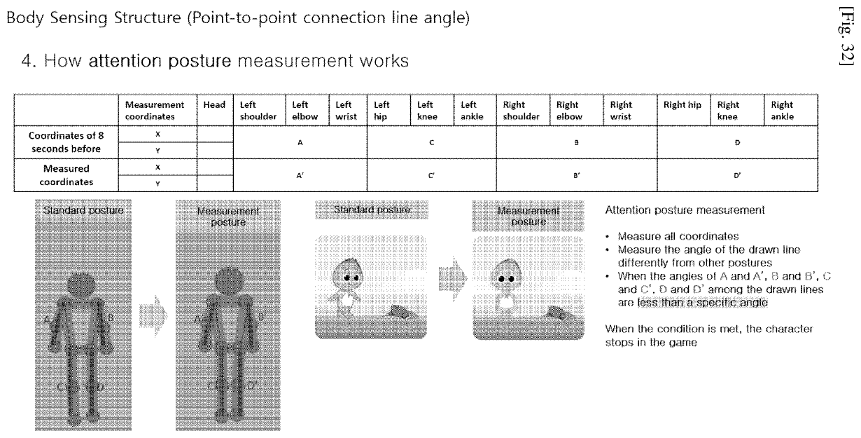

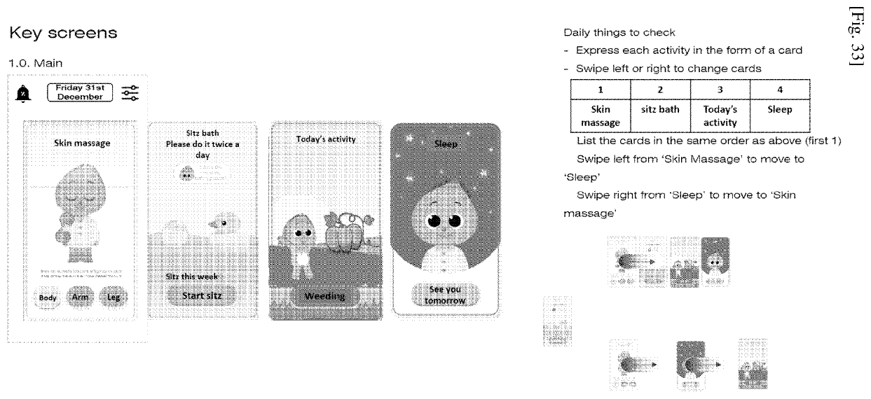

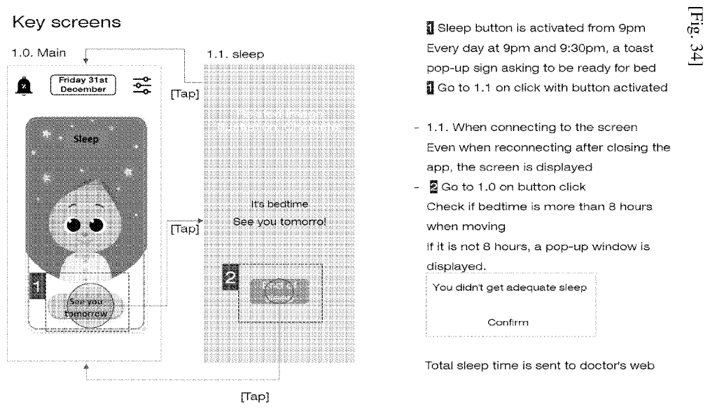

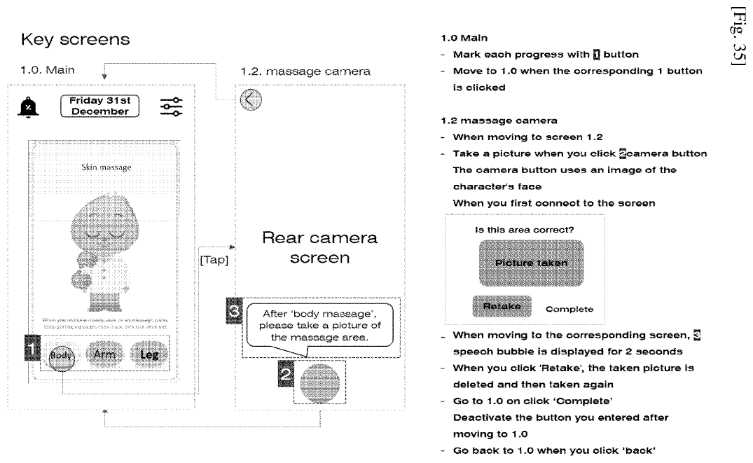

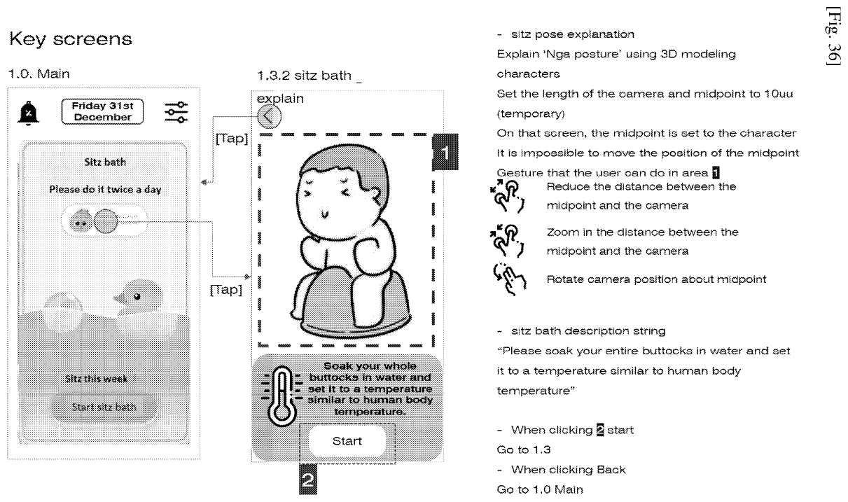

Exemplary screens for skin massage, sitz bath, activity and sleep are shown in FIGS. 33-36.

### Certain Embodiments

Embodiment 1. A method of strengthening or increasing mass of a pelvic floor muscle in a subject, the method comprising: providing, by an electronic device to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising one or more first instructions for the subject to follow.

Embodiment 2. The method according to Embodiment 1, wherein the electronic device (i) comprises a sensor for sensing adherence by the subject to the first instructions of the one or more first modules, (ii) transmits adherence information, based on the adherence, to a server, and (iii) receives one or more second instructions from the server based on the adherence information.

Embodiment 3. The method according to Embodiment 1 or 2, further comprising providing, by the electronic device to the subject, one or more second modules selected from the group consisting of an exercise and/or life style module, a sitz bath module, a pelvic splanchnic nerve and/or pelvic muscle exercise module, and a vagal nerve system and/or gastrointestinal (GI) exercise module, the one or more second modules comprising the one or more second instructions.

Embodiment 4. The method according to any one of Embodiments 1-3, wherein the one or more first modules or the one or more second modules comprise the exercise and/or life style module, and the exercise and/or life style module comprises one or more first instructions to increase a secretion of at least one of growth hormone (GH) and insulin-like growth factor-1 (IGF-1) in the subject.

Embodiment 5. The method according to any one of Embodiments 1-4, wherein the exercise and/or life style module comprises one or more first instructions for aerobic, resistance, and/or concurrent exercise.

Embodiment 6. The method according to Embodiment 5, wherein the instructions for the aerobic, the resistance, and/or the concurrent exercise comprise instructions for one or more independently selected from the group consisting of gripping a finger, tummy time, lifting weights, moving toe, moving ear, and a physical activity.

Embodiment 7. The method according to Embodiment 6, wherein the physical activity is selected from the group consisting of a structured exercise, kickball, riding a pedal-driven vehicle, jumping, stretching, playing a ball game, skeletal muscle exercise, walking, and aerobic and resistance exercise.

Embodiment 8. The method according to any one of Embodiments 1-7, wherein the one or more first instructions comprise instructions for obtaining one or more of deep sleep and adequate sleep.

Embodiment 9. The method according to any one of Embodiments 1-8, wherein the one or more first modules or the one or more second modules comprise the sitz bath module, and the sitz bath module comprises one or more first instructions to maintain non-ischemic status thereby stimulating myosin formation, stimulate rectal ENS maturation, and/or stimulate GLP2 secretion for rectal endocrine cells.

Embodiment 10. The method according to any one of Embodiments 1-9, wherein the sitz bath module, and the sitz bath module comprises one or more first instructions to take warm bath, hip bath and/or sitz bath.

Embodiment 11. The method according to any one of Embodiments 1-10, wherein the one or more first modules or the one or more second modules comprise the pelvic splanchnic nerve and/or pelvic muscle exercise module, and the pelvic splanchnic nerve and/or pelvic muscle exercise module comprises one or more first instructions to stimulate rectal ENS maturation, and/or stimulate GLP2 secretion for rectal endocrine cells.

Embodiment 12. The method according to any one of Embodiments 1-11, wherein the pelvic splanchnic nerve and/or pelvic muscle exercise module comprises one or more first instructions for cycling away, performing a wheelbarrow, walking, bar hanging, standing upright, trying to touch ceiling, freezing movement, and running.

Embodiment 13. The method according to any one of Embodiments 1-12, wherein the one or more first modules or the one or more second modules comprise the vagal nerve system and/or GI exercise module, and the vagal nerve system and/or GI exercise module comprises one or more first instructions to stimulate rectal and/or GI ENS maturation, stimulate GLP2 secretion for rectal and/or GI endocrine cells, and/or decrease inflammation signaling factors.

Embodiment 14. The method according to any one of Embodiments 1-13, wherein the one or more first modules or the one or more second modules comprise the vagal nerve system and/or GI exercise module, and the vagal nerve system and/or GI exercise module comprises one or more first instructions to improve anorectal motility.

Embodiment 15. The method according to Embodiment 14, wherein the one or more first instructions to improve anorectal motility are selected from the group consisting of aerobic exercises including walking, running, cycling, and swimming, walking hand-in-hand with another individual, bar hanging, standing upright, reaching for the ceiling, and limiting motion/playing a freeze game, playing with a small animal, high-intensity playing, adjusting posture, and tapping on a surface.

Embodiment 16. The method according to any one of Embodiments 1-15, wherein the vagal nerve system and/or GI exercise module comprises at least one instructions selected from the group consisting of sense stimulation instructions for sight, sound, touch, taste, and smell.

Embodiment 17. The method according to Embodiment 16, wherein the vagal nerve stimulation module comprises one or more sense stimulation instructions for sight, and the one or more sense stimulation instructions for sight include one or more instructions to view one or more figures to stimulate autonomic nervous system.

Embodiment 18. The method according to Embodiment 17, wherein the one or more figures to stimulate the autonomic nervous system are selected from the group consisting of erotic photos, photos of cold, photos of heat, and frightening photos.

Embodiment 19. The method according to Embodiment 17 or 18, wherein the electronic device receives and displays the one or more figures.

Embodiment 20. The method according to any one of Embodiments 16-19, wherein the vagal nerve stimulation module comprises one or more sense stimulation instructions for sound, and the one or more sense stimulation instructions for sound include one or more instructions to hear one or more sounds to cause honor or re laxation.

Embodiment 21. The method according to Embodiment 20, wherein the one or more instructions to hear one or more sounds to cause horror or relaxation are independently selected from the group consisting of frightening sounds, sounds capable of inducing chill or changes in the skin, raindrop sounds, and sounds capable of inducing sleep.

Embodiment 22. The method according to Embodiment 20 or 21, wherein the electronic device receives and plays the one or more sounds.

Embodiment 23. The method according to any one of Embodiments 16-22, wherein the vagal nerve stimulation module comprises one or more sense stimulation instructions for touch, and the one or more sense stimulation instructions for touch include one or more instructions for abdominal breathing, controlling rate of breathing, restricting breath for a period of time, controlling heart rate, controlling movement of the GI tract, cold massage, coughing, and skin massage.

Embodiment 24. The method according to any one of Embodiments 16-23, wherein the vagal nerve stimulation module comprises one or more sense stimulation instructions for taste, and the one or more sense stimulation instructions for taste include one or more instructions to eat food to stimulate digestive glands in the subject.

Embodiment 25. The method according to Embodiment 24, wherein the electronic device receives and display information related to the food.

Embodiment 26. The method according to any one of Embodiments 16-25, wherein the vagal nerve stimulation module comprises one or more sense stimulation instructions for smell, and the one or more sense stimulation instructions for smell include one or more instructions to stimulate digestive glands and/or to relax.

Embodiment 27. The method according to Embodiment 26, wherein the one or more instructions to stimulate digestive glands and/or to relax stimulate the autonomic nervous system, induce secretion of saliva in the subject, and/or reduce a heart rate of the subject.

Embodiment 28. The method according to Embodiment 26 or 27, wherein the electronic device is configured to release a scent for aroma therapy.

Embodiment 29. The method of any one of Embodiments 13-28, wherein the subject has Irritable Bowel Syndrome (IBS), and a treatment mechanism of the module comprises anti-inflammation.

Embodiment 30. The method of any one of Embodiments 13-28, wherein the subject has Inflammatory Bowel Disease (IBS), and a treatment mechanism of the module comprises one or both of anti-inflammation and motility adaptation.

Embodiment 31. The method of any one of Embodiments 13-28, wherein the subject has acute constipation, and a treatment mechanism of the module comprises motility adaptation.

Embodiment 32. The method of any one of Embodiments 13-28, wherein the subject has chronic constipation, and a treatment mechanism of the module comprises one or more of motility adaptation, biofeedback exercise, and blood circulation.

Embodiment 33. The method of Embodiment 32, wherein the one or more first modules or the one or more second modules further comprise one or both of an anorectal biofeedback module and a sitz bath.

Embodiment 34. The method of any one of Embodiments 13-28, wherein the subject has Necrotizing EnteroColitis (NEC), and a treatment mechanism of the module comprises anti-inflammation.

Embodiment 35. The method of any one of Embodiments 13-28, wherein the subject has an Adult-GI-post-operation recovery (anti-emesis/nausea, vomiting, no-gas out), and a treatment mechanism of the module comprises one or both of anti-inflammation and motility adaptation.

Embodiment 36. The method according to any one of Embodiments 1-35, wherein the subject is a post-operative patient with lower gastrointestinal-operable-congenital anomaly.

Embodiment 37. The method according to Embodiment 36, wherein the subject has had one or more operations selected from the group consisting of a pull-through surgery, an anorectoplasty, and anoplasty.

Embodiment 38. The method according to Embodiment 36 or 37, wherein, prior to or after having the operation, the subject has a condition selected from the group consisting of Hirschsprung disease, an anorectal malformation, and a meningocele.

Embodiment 39. The method according to any one of Embodiments 1-38, wherein an age of the subject is 3 months or older.

Embodiment 40. The method according to any one of Embodiments 1-39, wherein the method is first performed about 1 week after the operation.

Embodiment 41. The method according to any one of Embodiments 1-40, wherein the method is performed until the subject is about 20 years old.

Embodiment 42. The method according to any one of Embodiments 1-41, wherein the one or more first modules are provided to a guardian of the subject, and the subject received the modules indirectly from the electronic device through the guardian.

Embodiment 43. The method according to any one of Embodiments 1-42, wherein the one or more first modules consists of the exercise and/or life style module, the sitz bath module, the pelvic splanchnic nerve and/or pelvic muscle exercise module, and the vagal nerve system and/or gastrointestinal (GI) exercise module.

Embodiment 44. The method according to any one of Embodiments 2-43, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 45. The method according to any one of Embodiments 2-44, wherein the sensor comprises a touch sensor, and the subject provides the adherence in formation to the electronic device using the touch sensor.

Embodiment 46. The method according to any one of Embodiments 2-45, wherein the sensor is a detachable peripheral.

Embodiment 47. The method according to any one of Embodiments 2-46, wherein the adherence information includes changes of coordinates on at least two points on the subject over time.

Embodiment 48. The method according to any one of Embodiments 2-47, wherein the adherence information includes changes of coordinates on at least ten points on the subject over time.

Embodiment 49. The method according to Embodiment 47 or 48, wherein said points are on positions selected from the group consisting of head, shoulder, elbow, wrist, waist, knee, and ankle of the subject.

Embodiment 50. The method according to any one of Embodiments 47-49, wherein the first instructions comprise changes of coordinates on said points, and the changes of coordinates of the adherence information are compared with the changes of co ordinates of the first instructions.

Embodiment 51. A system for strengthening or increasing mass of a pelvic floor muscle in a subject, comprising: a digital apparatus configured to execute a digital application comprising one or more first modules, for strengthening or increasing mass of a pelvic floor muscle in a subject, wherein the digital apparatus comprises a sensor for sensing adherence by the subject to a first set of instructions of the one or more first modules; a healthcare provider portal configured to provide one or more options to a healthcare provider to perform one or more tasks to prescribe treatment to strengthen or increase mass of a pelvic floor muscle in the subject based on information received from the digital application; and an administrative portal configured to provide one or more options to an administrator of the system to perform one or more tasks to manage access to the system by the healthcare provider.

Embodiment 52. The system according to Embodiment 51, wherein the digital apparatus (i) comprises a sensor for sensing adherence by the subject to the first instructions of the one or more first modules, (ii) transmits adherence information, based on the adherence, to a server, and (iii) receives one or more second instructions from the server based on the adherence information.

Embodiment 53. The system according to Embodiment 51 or 52, further comprising providing, by the digital apparatus to the subject, one or more second modules selected from the group consisting of an exercise and/or life style module, a sitz bath module, a pelvic splanchnic nerve and/or pelvic muscle exercise module, and a vagal nerve system and/or gastrointestinal (GI) exercise module, the one or more second modules comprising the one or more second instructions.

Embodiment 54. The system according to any one of Embodiments 51-53 wherein the one or more first modules or the one or more second modules comprise the exercise and/or life style module, and the exercise and/or life style module comprises one or more first instructions to increase a secretion of at least one of growth hormone (GH) and insulin-like growth factor-1 (IGF-1) in the subject.

Embodiment 55. The system according to any one of Embodiments 51-54, wherein the exercise and/or life style module comprises one or more first instructions for aerobic, resistance, and/or concurrent exercise.

Embodiment 56. The system according to Embodiment 55, wherein the instructions for the aerobic, the resistance, and/or the concurrent exercise comprise instructions for one or more independently selected from the group consisting of gripping a finger, tummy time, lifting weights, moving toe, moving ear, and a physical activity

Embodiment 57. The system according to Embodiment 56, wherein the physical activity is selected from the group consisting of a structured exercise, kickball, riding a pedal-driven vehicle, jumping, stretching, playing a ball game, skeletal muscle exercise, walking, and aerobic and resistance exercise.

Embodiment 58. The system according to any one of Embodiments 51-57, wherein the one or more first instructions comprise instructions for obtaining one or more of deep sleep and adequate sleep.

Embodiment 59. The system according to any one of Embodiments 51-58, wherein the one or more first modules or the one or more second modules comprise the sitz bath module, and the sitz bath module comprises one or more first instructions to maintain non-ischemic status thereby stimulating myosin formation, stimulate rectal ENS maturation, and/or stimulate GLP2 secretion for rectal endocrine cells.

Embodiment 60. The system according to any one of Embodiments 51-59, wherein the sitz bath module, and the sitz bath module comprises one or more first instructions to take warm bath, hip bath and/or sitz bath.

Embodiment 61. The system according to any one of Embodiments 51-60, wherein the one or more first modules or the one or more second modules comprise the pelvic splanchnic nerve and/or pelvic muscle exercise module, and the pelvic splanchnic nerve and/or pelvic muscle exercise module comprises one or more first instructions to stimulate rectal ENS maturation, and/or stimulate GLP2 secretion for rectal endocrine cells.

Embodiment 62. The system according to any one of Embodiments 51-56, wherein the pelvic splanchnic nerve and/or pelvic muscle exercise module comprises one or more first instructions for cycling away, performing a wheelbarrow, walking, bar hanging, standing upright, trying to touch ceiling, freezing movement, and running.

Embodiment 63. The system according to any one of Embodiments 51-62, wherein the one or more first modules or the one or more second modules comprise the vagal nerve system and/or GI exercise module, and the vagal nerve system and/or GI exercise module comprises one or more first instructions to stimulate rectal and/or GI ENS maturation, stimulate GLP2 secretion for rectal and/or GI endocrine cells, and/or decrease inflammation signaling factors.

Embodiment 64. The system according to any one of Embodiments 51-63, wherein the one or more first modules or the one or more second modules comprise the vagal nerve system and/or GI exercise module, and the vagal nerve system and/or GI exercise module comprises one or more first instructions to improve anorectal motility.

Embodiment 65. The system according to Embodiment 64, wherein the one or more first instructions to improve anorectal motility are selected from the group consisting of aerobic exercises including walking, running, cycling, and swimming, walking hand-in-hand with another individual, bar hanging, standing upright, reaching for the ceiling, and limiting motion/playing a freeze game, playing with a small animal, high-intensity playing, adjusting posture, and tapping on a surface.

Embodiment 66. The system according to any one of Embodiments 51-65, wherein the vagal nerve system and/or GI exercise module comprises at least one instructions selected from the group consisting of sense stimulation instructions for sight, sound, touch, taste, and smell.

Embodiment 67. The system according to Embodiment 66, wherein the vagal nerve stimulation module comprises one or more sense stimulation instructions for sight, and the one or more sense stimulation instructions for sight include one or more instructions to view one or more figures to stimulate autonomic nervous system.

Embodiment 68. The system according to Embodiment 67, wherein the one or more figures to stimulate the autonomic nervous system are selected from the group consisting of erotic photos, photos of cold, photos of heat, and frightening photos.

Embodiment 69. The system according to Embodiment 66 or 67, wherein the digital apparatus receives and displays the one or more figures.

Embodiment 70. The system according to any one of Embodiments 66-69, wherein the vagal nerve stimulation module comprises one or more sense stimulation instructions for sound, and the one or more sense stimulation instructions for sound include one or more instructions to hear one or more sounds to cause honor or re laxation.

Embodiment 71. The system according to Embodiment 70, wherein the one or more instructions to hear one or more sounds to cause horror or relaxation are independently selected from the group consisting of frightening sounds, sounds capable of inducing chill or changes in the skin, raindrop sounds, and sounds capable of inducing sleep.

Embodiment 72. The system according to Embodiment 70 or 71, wherein the digital apparatus receives and plays the one or more sounds.

Embodiment 73. The system according to any one of Embodiments 66-72, wherein the vagal nerve stimulation module comprises one or more sense stimulation instructions for touch, and the one or more sense stimulation instructions for touch include one or more instructions for abdominal breathing, controlling rate of breathing, restricting breath for a period of time, controlling heart rate, controlling movement of the GI tract, cold massage, coughing, and skin massage.

Embodiment 74. The system according to any one of Embodiments 66-73, wherein the vagal nerve stimulation module comprises one or more sense stimulation instructions for taste, and the one or more sense stimulation instructions for taste include one or more instructions to eat food to stimulate digestive glands in the subject.

Embodiment 75. The system according to Embodiment 74, wherein the digital apparatus receives and display information related to the food.

Embodiment 76. The system according to any one of Embodiments 66-75, wherein the vagal nerve stimulation module comprises one or more sense stimulation instructions for smell, and the one or more sense stimulation instructions for smell include one or more instructions to stimulate digestive glands and/or to relax.

Embodiment 77. The system according to Embodiment 76, wherein the one or more instructions to stimulate digestive glands and/or to relax stimulate the autonomic nervous system, induce secretion of saliva in the subject, and/or reduce a heart rate of the subject.

Embodiment 78. The system according to Embodiment 76 or 77, wherein the digital apparatus is configured to release a scent for aroma therapy.

Embodiment 79. The system according to any one of Embodiments 63-78, wherein the subject has Irritable Bowel Syndrome (IBS), and a treatment mechanism of the module comprises anti-inflammation.

Embodiment 80. The system according to any one of Embodiments 63-78, wherein the subject has Inflammatory Bowel Disease (IBS), and a treatment mechanism of the module comprises one or both of anti-inflammation and motility adaptation.

Embodiment 81. The system according to any one of Embodiments 63-78, wherein the subject has acute constipation, and a treatment mechanism of the module comprises motility adaptation.

Embodiment 82. The system according to any one of Embodiments 63-78, wherein the subject has chronic constipation, and a treatment mechanism of the module comprises one or more of motility adaptation, biofeedback exercise, and blood circulation.

Embodiment 83. The system according to Embodiment 82, wherein the one or more first modules or the one or more second modules further comprise one or both of an anorectal biofeedback module and a sitz bath.

Embodiment 84. The system according to any one of Embodiments 63-78, wherein the subject has Necrotizing EnteroColitis (NEC), and a treatment mechanism of the module comprises anti-inflammation.

Embodiment 85. The system according to any one of Embodiments 63-78, wherein the subject has an Adult-GI-post-operation recovery (anti-emesis/nausea, vomiting, no-gas out), and a treatment mechanism of the module comprises one or both of anti-inflammation and motility adaptation.

Embodiment 86. The system according to any one of Embodiments 51-85, wherein the subject is a post-operative patient with lower gastrointestinal-operable-congenital anomaly.

Embodiment 87. The system according to Embodiment 86, wherein the subject has had one or more operations selected from the group consisting of a pull-through surgery, an anorectoplasty, and anoplasty.

Embodiment 88. The system according to Embodiment 86 or 87, wherein, prior to or after having the operation, the subject has a condition selected from the group consisting of Hirschsprung disease, an anorectal malformation, and a meningocele.

Embodiment 89. The system according to any one of Embodiments 51-88, wherein an age of the subject is 3 months or older.

Embodiment 90. The system according to any one of Embodiments 51-89, wherein the module is first performed about 1 week after the operation.

Embodiment 91. The system according to any one of Embodiments 51-90, wherein the module is performed until the subject is about 20 years old.

Embodiment 92. The system according to any one of Embodiments 51-91, wherein the one or more first modules are provided to a guardian of the subject, and the subject received the modules indirectly from the electronic device through the guardian.

Embodiment 93. The system according to any one of Embodiments 51-92, wherein the one or more first modules consists of the exercise and/or life style module, the sitz bath module, the pelvic splanchnic nerve and/or pelvic muscle exercise module, and the vagal nerve system and/or gastrointestinal (GI) exercise module.

Embodiment 94. The system according to any one of Embodiments 51-93, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 95. The system according to any one of Embodiments 51-94, wherein the sensor comprises a touch sensor, and the subject provides the adherence in formation to the electronic device using the touch sensor.

Embodiment 96. The system according to any one of Embodiments 51-95, wherein the sensor is a detachable peripheral.

Embodiment 97. The system according to any one of Embodiments 51-96, wherein the adherence information includes changes of coordinates on at least two points on the subject over time.

Embodiment 98. The system according to any one of Embodiments 51-97, wherein the adherence information includes changes of coordinates on at least ten points on the subject over time.

Embodiment 99. The system according to Embodiment 97 or 98, wherein said points are on positions selected from the group consisting of head, shoulder, elbow, wrist, waist, knee, and ankle of the subject.

Embodiment 100. The system according to any one of Embodiments 97-99, wherein the first instructions comprise changes of coordinates on said points, and the changes of coordinates of the adherence information are compared with the changes of co ordinates of the first instructions.

Embodiment 101. The system according to any one of Embodiment 51-100, wherein the one or more options provided to the healthcare provider are selected from the group consisting of adding or removing the subject, viewing or editing personal information for the subject, viewing adherence information for the subject, viewing a result of the subject for one or more at least partially completed modules, prescribing one or more modules to the subject, altering a prescription for one or more modules, and communicating with the subject.

Embodiment 102. The system according to Embodiment 101, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.

Embodiment 103. The system according to Embodiment 101, wherein the personal information comprises the prescription for the subject, and the prescription for the subject comprises one or more selected from the group consisting of a prescription identification number, a prescription type, a start date, a duration, a completion date, a number of scheduled or prescribed modules to be performed by the subject, and a number of scheduled or prescribed modules to be performed by the subject per day.

Embodiment 104. The system according to any one of Embodiments 101-103, wherein the one or more options comprise the viewing the adherence information, and the adherence information of the subject comprises one or more of a number of scheduled or prescribed modules completed by the subject, and a calendar identifying one or more days on which the subject completed, partially completed, or did not complete one or more scheduled or prescribed modules.

Embodiment 105. The system according to any one of Embodiments 101-104, wherein the one or more options comprise the viewing the result of the subject, and the result of the subject for one or more at least partially completed modules comprises one or more selected from the group consisting of a time at which the subject started a scheduled or prescribed module, a time at which the subject ended a scheduled or prescribed module, and an indicator of whether the scheduled or prescribed module was fully or partially completed.

Embodiment 106. The system according to Embodiment 51-105, wherein the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed modules, and communicating with the healthcare provider.

Embodiment 107. The system according to Embodiment 106, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.

Embodiment 108. The system according to Embodiment 106 or 107, wherein the one or more options comprise the viewing or editing the de-identified information of the subject, and the de-identified information of the subject comprises one or more selected from the group consisting of an identification number for the subject, and the healthcare provider for the subject.

Embodiment 109. The system according to any one of Embodiments 106-108, wherein the one or more options comprise the viewing the adherence information for the subject, and the adherence information of the subject comprises one or more of a number of scheduled or prescribed modules completed by the subject, and a calendar identifying one or more days on which the subject completed, partially completed, or did not complete one or more scheduled or prescribed modules.

Embodiment 110. The system according to any one of Embodiments 101-109, wherein the one or more options comprise the viewing the result of the subject, and the result of the subject for one or more at least partially completed modules comprises one or more selected from the group consisting of a time at which the subject started a scheduled or prescribed module, a time at which the subject ended a scheduled or prescribed module, and an indicator of whether the scheduled or prescribed module was fully or partially completed.

Embodiment 111. The system according to any one of Embodiments 51-110, wherein the digital application further comprises a push alarm for one or more of reminding the subject complete a module.

Embodiment 112. The system according to any one of Embodiments 51-111, wherein the digital apparatus comprises: a digital instruction generation unit configured to generate modules for strengthening or increasing mass of a pelvic floor muscle in a subject, generate digital instructions based on the modules, and provide the digital instructions to the subject; and an outcome collection unit configured to collect the subject's execution outcomes of the digital instructions.

Embodiment 113. The system according to any one of Embodiments 51-112, wherein the digital instruction generation unit generates the modules based on the inputs from the healthcare provider.

Embodiment 114. The system according to any one of Embodiments 51-113, wherein the digital instruction generation unit generates the modules based on in formation received from the subject.

Embodiment 115. The system of Embodiment 114, wherein the information is received from the subject comprises at least one of basal factors, medical information, and digital therapeutics literacy of the subject, the basal factors including the subject's activity, heart rate, sleep, and diet (including nutrition and calories), the medical in formation including the subject's electronic medical record (EMR), family history, genetic vulnerability, and genetic susceptibility, and the digital therapeutics literacy including the subject's accessibility, and technology adoption to the digital therapeutics and the apparatus.

Embodiment 116. The system according to any one of Embodiments 51-115, wherein the digital instruction generation unit generates the modules matching to imaginary parameters which correspond to the mechanism of action in and the therapeutic hypothesis for a lower gastrointestinal-operable-congenital anomaly in the subject.

Embodiment 117. The system of Embodiment 116, wherein the imaginary parameters are deduced in relation to the subject's environment, behaviors, emotions, and cognition.

Embodiment 118. The system according to any one of Embodiments 51-117, wherein the outcome collection unit collects the execution outcomes of the digital instructions by monitoring the subject's adherence to the digital instructions or allowing the subject to directly input the subject's adherence to the digital instructions.

Embodiment 119. The system according to any one of Embodiments 51-118, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

Embodiment 120. A computing system for strengthening or increasing mass of a pelvic floor muscle in a subject in need thereof, comprising: a display configured to provide, to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising one or more first instructions for the subject to follow; a sensor configured to sense adherence by the subject to the instructions of the one or more first modules; a transmitter configured to transmit adherence information, based on the adherence, to a server; and a receiver configured to receive, from the server, one or more second instructions based on the adherence information.

Embodiment 121. The computing system according to Embodiment 120, further comprising providing, by the computing system to the subject, one or more second modules selected from the group consisting of an exercise and/or life style module, a sitz bath module, a pelvic splanchnic nerve and/or pelvic muscle exercise module, and a vagal nerve system and/or gastrointestinal (GI) exercise module, the one or more second modules comprising the one or more second instructions.

Embodiment 122. The computing system according to Embodiment 120 or 121, wherein the one or more first modules or the one or more second modules comprise the exercise and/or life style module, and the exercise and/or life style module comprises one or more first instructions to increase a secretion of at least one of growth hormone (GH) and insulin-like growth factor-1 (IGF-1) in the subject.

Embodiment 123. The computing system according to any one of Embodiments 120-122, wherein the exercise and/or life style module comprises one or more first instructions for aerobic, resistance, and/or concurrent exercise.

Embodiment 124. The computing system according to Embodiment 123, wherein the instructions for the aerobic, the resistance, and/or the concurrent exercise comprise instructions for one or more independently selected from the group consisting of gripping a finger, tummy time, lifting weights, moving toe, moving ear, and a physical activity.

Embodiment 125. The computing system according to Embodiment 124, wherein the physical activity is selected from the group consisting of a structured exercise, kickball, riding a pedal-driven vehicle, jumping, stretching, playing a ball game, skeletal muscle exercise, walking, and aerobic and resistance exercise.

Embodiment 126. The computing system according to any one of Embodiments 120-125, wherein the one or more first instructions comprise instructions for obtaining one or more of deep sleep and adequate sleep.

Embodiment 127. The computing system according to any one of Embodiments 120-126, wherein the one or more first modules or the one or more second modules comprise the sitz bath module, and the sitz bath module comprises one or more first instructions to maintain non-ischemic status thereby stimulating myosin formation, stimulate rectal ENS maturation, and/or stimulate GLP2 secretion for rectal endocrine cells.

Embodiment 128. The computing system according to any one of Embodiments 120-127, wherein the sitz bath module, and the sitz bath module comprises one or more first instructions to take warm bath, hip bath and/or sitz bath.

Embodiment 129. The computing system according to any one of Embodiments 120-128, wherein the one or more first modules or the one or more second modules comprise the pelvic splanchnic nerve and/or pelvic muscle exercise module, and the pelvic splanchnic nerve and/or pelvic muscle exercise module comprises one or more first instructions to stimulate rectal ENS maturation, and/or stimulate GLP2 secretion for rectal endocrine cells.

Embodiment 130. The computing system according to any one of Embodiments 120-129, wherein the pelvic splanchnic nerve and/or pelvic muscle exercise module comprises one or more first instructions for cycling away, performing a wheelbarrow, walking, bar hanging, standing upright, trying to touch ceiling, freezing movement, and running.

Embodiment 131. The computing system according to any one of Embodiments 120-130, wherein the one or more first modules or the one or more second modules comprise the vagal nerve system and/or GI exercise module, and the vagal nerve system and/or GI exercise module comprises one or more first instructions to stimulate rectal and/or GI ENS maturation, stimulate GLP2 secretion for rectal and/or GI endocrine cells, and/or decrease inflammation signaling factors.

Embodiment 132. The computing system according to any one of Embodiments 120-131, wherein the one or more first modules or the one or more second modules comprise the vagal nerve system and/or GI exercise module, and the vagal nerve system and/or GI exercise module comprises one or more first instructions to improve anorectal motility.

Embodiment 133. The computing system according to Embodiment 132, wherein the one or more first instructions to improve anorectal motility are selected from the group consisting of aerobic exercises including walking, running, cycling, and swimming, walking hand-in-hand with another individual, bar hanging, standing upright, reaching for the ceiling, and limiting motion/playing a freeze game, playing with a small animal, high-intensity playing, adjusting posture, and tapping on a surface.

Embodiment 134. The computing system according to any one of Embodiments 120-133, wherein the vagal nerve system and/or GI exercise module comprises at least one instructions selected from the group consisting of sense stimulation instructions for sight, sound, touch, taste, and smell.

Embodiment 135. The computing system according to Embodiment 134, wherein the vagal nerve stimulation module comprises one or more sense stimulation instructions for sight, and the one or more sense stimulation instructions for sight include one or more instructions to view one or more figures to stimulate autonomic nervous system.

Embodiment 136. The computing system according to Embodiment 135, wherein the one or more figures to stimulate the autonomic nervous system are selected from the group consisting of erotic photos, photos of cold, photos of heat, and frightening photos.

Embodiment 137. The computing system according to Embodiment 135 or 136, wherein the computing system receives and displays the one or more figures.

Embodiment 138. The computing system according to any one of Embodiments 134-137, wherein the vagal nerve stimulation module comprises one or more sense stimulation instructions for sound, and the one or more sense stimulation instructions for sound include one or more instructions to hear one or more sounds to cause horror or relaxation.

Embodiment 139. The computing system according to Embodiment 138, wherein the one or more instructions to hear one or more sounds to cause horror or relaxation are independently selected from the group consisting of frightening sounds, sounds capable of inducing chill or changes in the skin, raindrop sounds, and sounds capable of inducing sleep.

Embodiment 140. The computing system according to Embodiment 138 or 139, wherein the computing system receives and plays the one or more sounds.

Embodiment 141. The computing system according to any one of Embodiments 134-140, wherein the vagal nerve stimulation module comprises one or more sense stimulation instructions for touch, and the one or more sense stimulation instructions for touch include one or more instructions for abdominal breathing, controlling rate of breathing, restricting breath for a period of time, controlling heart rate, controlling movement of the GI tract, cold massage, coughing, and skin massage.

Embodiment 142. The computing system according to any one of Embodiments 134-141, wherein the vagal nerve stimulation module comprises one or more sense stimulation instructions for taste, and the one or more sense stimulation instructions for taste include one or more instructions to eat food to stimulate digestive glands in the subject.

Embodiment 143. The computing system according to Embodiment 142, wherein the computing system receives and display information related to the food.

Embodiment 144. The computing system according to any one of Embodiments 134-143, wherein the vagal nerve stimulation module comprises one or more sense stimulation instructions for smell, and the one or more sense stimulation instructions for smell include one or more instructions to stimulate digestive glands and/or to relax.

Embodiment 145. The computing system according to Embodiment 144, wherein the one or more instructions to stimulate digestive glands and/or to relax stimulate the autonomic nervous system, induce secretion of saliva in the subject, and/or reduce a heart rate of the subject.

Embodiment 146. The computing system according to Embodiment 144 or 145, wherein the computing system is configured to release a scent for aroma therapy.

Embodiment 147. The computing system according to any one of Embodiments 131-146, wherein the subject has Irritable Bowel Syndrome (IBS), and a treatment mechanism of the module comprises anti-inflammation.

Embodiment 148. The computing system according to any one of Embodiments 131-146, wherein the subject has Inflammatory Bowel Disease (IBS), and a treatment mechanism of the module comprises one or both of anti-inflammation and motility adaptation.

Embodiment 149. The computing system according to any one of Embodiments 131-146, wherein the subject has acute constipation, and a treatment mechanism of the module comprises motility adaptation.

Embodiment 150. The computing system according to any one of Embodiments 131-146, wherein the subject has chronic constipation, and a treatment mechanism of the module comprises one or more of motility adaptation, biofeedback exercise, and blood circulation.

Embodiment 151. The computing system according to Embodiment 150, wherein the one or more first modules or the one or more second modules further comprise one or both of an anorectal biofeedback module and a sitz bath.

Embodiment 152. The computing system according to any one of Embodiments 131-146, wherein the subject has Necrotizing EnteroColitis (NEC), and a treatment mechanism of the module comprises anti-inflammation.

Embodiment 153. The computing system of any one of Embodiments 131-146, wherein the subject has an Adult-GI-post-operation recovery (anti-emesis/nausea, vomiting, no-gas out), and a treatment mechanism of the module comprises one or both of anti-inflammation and motility adaptation.

Embodiment 154. The computing system according to any one of Embodiments 120-153, wherein the subject is a post-operative patient with lower gastrointestinal-operable-congenital anomaly.

Embodiment 155. The computing system according to Embodiment 154, wherein the subject has had one or more operations selected from the group consisting of a pull-through surgery, an anorectoplasty, and anoplasty.

Embodiment 156. The computing system according to Embodiment 154 or 155, wherein, prior to or after having the operation, the subject has a condition selected from the group consisting of Hirschsprung disease, an anorectal malformation, and a meningocele.

Embodiment 157. The computing system according to any one of Embodiments 120-156, wherein an age of the subject is 3 months or older.

Embodiment 158. The computing system according to any one of Embodiments 120-157, wherein the module is first performed about 1 week after the operation.

Embodiment 159. The computing system according to any one of Embodiments 120-158, wherein the module is performed until the subject is about 20 years old.

Embodiment 160. The computing system according to any one of Embodiments 120-159, wherein the one or more first modules are provided to a guardian of the subject, and the subject received the modules indirectly from the computing system through the guardian.

Embodiment 161. The computing system according to any one of Embodiments 120-160, wherein the one or more first modules consists of the exercise and/or life style module, the sitz bath module, the pelvic splanchnic nerve and/or pelvic muscle exercise module, and the vagal nerve system and/or gastrointestinal (GI) exercise module.

Embodiment 162. The computing system according to any one of Embodiments 120-161, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 163. The computing system according to any one of Embodiments 120-162, wherein the sensor comprises a touch sensor, and the subject provides the adherence information to the computing system using the touch sensor.

Embodiment 164. The computing system according to any one of Embodiments 120-163, wherein the sensor is a detachable peripheral.

Embodiment 165. The computing system according to any one of Embodiments 120-164, wherein the adherence information includes changes of coordinates on at least two points on the subject over time.

Embodiment 166. The computing system according to any one of Embodiments 120-165, wherein the adherence information includes changes of coordinates on at least ten points on the subject over time.

Embodiment 167. The computing system according to Embodiment 165 or 166, wherein said points are on positions selected from the group consisting of head, shoulder, elbow, wrist, waist, knee, and ankle of the subject.

Embodiment 168. The computing system according to any one of Embodiments 165-167, wherein the first instructions comprise changes of coordinates on said points, and the changes of coordinates of the adherence information are compared with the changes of coordinates of the first instructions.

Embodiment 169. A non-transitory computer readable medium having stored thereon software instructions for strengthening or increasing mass of a pelvic floor muscle in a subject in need thereof that, when executed by a processor, cause the processor to: display, by an electronic device to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising instructions for the subject to follow; sense, by a sensor, adherence by the subject to the instructions of the one or more first modules; transmit, by the electronic device, adherence information, based on the adherence, to a server; and receive, from the server, one or more second instructions based on the adherence information.

Embodiment 170. The non-transitory computer readable medium according to Embodiment 169, further comprising providing, by the electronic device to the subject, one or more second modules selected from the group consisting of an exercise and/or life style module, a sitz bath module, a pelvic splanchnic nerve and/or pelvic muscle exercise module, and a vagal nerve system and/or gastrointestinal (GI) exercise module, the one or more second modules comprising the one or more second instructions.

Embodiment 171. The non-transitory computer readable medium according to Embodiment 169 or 170, wherein the one or more first modules or the one or more second modules comprise the exercise and/or life style module, and the exercise and/or life style module comprises one or more first instructions to increase a secretion of at least one of growth hormone (GH) and insulin-like growth factor-1 (IGF-1) in the subject.

Embodiment 172. The non-transitory computer readable medium according to any one of Embodiments 169-171, wherein the exercise and/or life style module comprises one or more first instructions for aerobic, resistance, and/or concurrent exercise.

Embodiment 173. The non-transitory computer readable medium according to Embodiment 172, wherein the instructions for the aerobic, the resistance, and/or the concurrent exercise comprise instructions for one or more independently selected from the group consisting of gripping a finger, tummy time, lifting weights, moving toe, moving ear, and a physical activity.

Embodiment 174. The non-transitory computer readable medium according to Embodiment 173 wherein the physical activity is selected from the group consisting of a structured exercise, kickball, riding a pedal-driven vehicle, jumping, stretching, playing a ball game, skeletal muscle exercise, walking, and aerobic and resistance exercise.

Embodiment 175. The non-transitory computer readable medium according to any one of Embodiments 169-174, wherein the one or more first instructions comprise instructions for obtaining one or more of deep sleep and adequate sleep.

Embodiment 176. The non-transitory computer readable medium according to any one of Embodiments 169-175, wherein the one or more first modules or the one or more second modules comprise the sitz bath module, and the sitz bath module comprises one or more first instructions to maintain non-ischemic status thereby stimulating myosin formation, stimulate rectal ENS maturation, and/or stimulate GLP2 secretion for rectal endocrine cells.

Embodiment 177. The non-transitory computer readable medium according to any one of Embodiments 169-176, wherein the sitz bath module, and the sitz bath module comprises one or more first instructions to take warm bath, hip bath and/or sitz bath.

Embodiment 178. The non-transitory computer readable medium according to any one of Embodiments 169-177, wherein the one or more first modules or the one or more second modules comprise the pelvic splanchnic nerve and/or pelvic muscle exercise module, and the pelvic splanchnic nerve and/or pelvic muscle exercise module comprises one or more first instructions to stimulate rectal ENS maturation, and/or stimulate GLP2 secretion for rectal endocrine cells.

Embodiment 179. The non-transitory computer readable medium according to any one of Embodiments 169-178, wherein the pelvic splanchnic nerve and/or pelvic muscle exercise module comprises one or more first instructions for cycling away, performing a wheelbarrow, walking, bar hanging, standing upright, trying to touch ceiling, freezing movement, and running.

Embodiment 180. The non-transitory computer readable medium according to any one of Embodiments 169-179, wherein the one or more first modules or the one or more second modules comprise the vagal nerve system and/or GI exercise module, and the vagal nerve system and/or GI exercise module comprises one or more first instructions to stimulate rectal and/or GI ENS maturation, stimulate GLP2 secretion for rectal and/or GI endocrine cells, and/or decrease inflammation signaling factors.

Embodiment 181. The non-transitory computer readable medium according to any one of Embodiments 169-180, wherein the one or more first modules or the one or more second modules comprise the vagal nerve system and/or GI exercise module, and the vagal nerve system and/or GI exercise module comprises one or more first instructions to improve anorectal motility.

Embodiment 182. The non-transitory computer readable medium according to Embodiment 181, wherein the one or more first instructions to improve anorectal motility are selected from the group consisting of aerobic exercises including walking, running, cycling, and swimming, walking hand-in-hand with another individual, bar hanging, standing upright, reaching for the ceiling, and limiting motion/playing a freeze game, playing with a small animal, high-intensity playing, adjusting posture, and tapping on a surface.

Embodiment 183. The non-transitory computer readable medium according to any one of Embodiments 169-182, wherein the vagal nerve system and/or GI exercise module comprises at least one instructions selected from the group consisting of sense stimulation instructions for sight, sound, touch, taste, and smell.

Embodiment 184. The non-transitory computer readable medium according to Embodiment 183, wherein the vagal nerve stimulation module comprises one or more sense stimulation instructions for sight, and the one or more sense stimulation instructions for sight include one or more instructions to view one or more figures to stimulate autonomic nervous system.

Embodiment 185. The non-transitory computer readable medium according to Embodiment 184, wherein the one or more figures to stimulate the autonomic nervous system are selected from the group consisting of erotic photos, photos of cold, photos of heat, and frightening photos.

Embodiment 186. The non-transitory computer readable medium according to Embodiment 184 or 185, wherein the electronic device receives and displays the one or more figures.

Embodiment 187. The non-transitory computer readable medium according to any one of Embodiments 183-186, wherein the vagal nerve stimulation module comprises one or more sense stimulation instructions for sound, and the one or more sense stimulation instructions for sound include one or more instructions to hear one or more sounds to cause horror or relaxation.

Embodiment 188. The non-transitory computer readable medium according to Embodiment 187, wherein the one or more instructions to hear one or more sounds to cause honor or relaxation are independently selected from the group consisting of frightening sounds, sounds capable of inducing chill or changes in the skin, raindrop sounds, and sounds capable of inducing sleep.

Embodiment 189. The non-transitory computer readable medium according to Embodiment 187 or 188, wherein the electronic device receives and plays the one or more sounds.

Embodiment 190. The non-transitory computer readable medium according to any one of Embodiments 183-189, wherein the vagal nerve stimulation module comprises one or more sense stimulation instructions for touch, and the one or more sense stimulation instructions for touch include one or more instructions for abdominal breathing, controlling rate of breathing, restricting breath for a period of time, con trolling heart rate, controlling movement of the GI tract, cold massage, coughing, and skin massage.

Embodiment 191. The non-transitory computer readable medium according to any one of Embodiments 183-190, wherein the vagal nerve stimulation module comprises one or more sense stimulation instructions for taste, and the one or more sense stimulation instructions for taste include one or more instructions to eat food to stimulate digestive glands in the subject.

Embodiment 192. The non-transitory computer readable medium according to Embodiment 191, wherein the electronic device receives and display information related to the food.

Embodiment 193. The non-transitory computer readable medium according to any one of Embodiments 183-192, wherein the vagal nerve stimulation module comprises one or more sense stimulation instructions for smell, and the one or more sense stimulation instructions for smell include one or more instructions to stimulate digestive glands and/or to relax.

Embodiment 194. The non-transitory computer readable medium according to Embodiment 193, wherein the one or more instructions to stimulate digestive glands and/or to relax stimulate the autonomic nervous system, induce secretion of saliva in the subject, and/or reduce a heart rate of the subject.

Embodiment 195. The non-transitory computer readable medium according to Embodiment 193 or 194, wherein the electronic device is configured to release a scent for aroma therapy.

Embodiment 196. The non-transitory computer readable medium according to any one of Embodiments 169-195, wherein the subject has Irritable Bowel Syndrome (IBS), and a treatment mechanism of the module comprises anti-inflammation.

Embodiment 197. The non-transitory computer readable medium according to any one of Embodiments 169-195, wherein the subject has Inflammatory Bowel Disease (IBS), and a treatment mechanism of the module comprises one or both of anti-inflammation and motility adaptation.

Embodiment 198. The non-transitory computer readable medium according to any one of Embodiments 169-195, wherein the subject has acute constipation, and a treatment mechanism of the module comprises motility adaptation.

Embodiment 199. The non-transitory computer readable medium according to any one of Embodiments 169-195, wherein the subject has chronic constipation, and a treatment mechanism of the module comprises one or more of motility adaptation, biofeedback exercise, and blood circulation.

Embodiment 200. The non-transitory computer readable medium according to any one of Embodiment 199, wherein the one or more first modules or the one or more second modules further comprise one or both of an anorectal biofeedback module and a sitz bath.

Embodiment 201. The non-transitory computer readable medium according to any one of Embodiments 169-195, wherein the subject has Necrotizing EnteroColitis (NEC), and a treatment mechanism of the module comprises anti-inflammation.

Embodiment 202. The non-transitory computer readable medium according to any one of Embodiments 169-195, wherein the subject has an Adult-GI-post-operation recovery (anti-emesis/nausea, vomiting, no-gas out), and a treatment mechanism of the module comprises one or both of anti-inflammation and motility adaptation.

Embodiment 203. The non-transitory computer readable medium according to any one of Embodiments 169-195, wherein the subject is a post-operative patient with lower gastrointestinal-operable-congenital anomaly.

Embodiment 204. The non-transitory computer readable medium according to Embodiment 203, wherein the subject has had one or more operations selected from the group consisting of a pull-through surgery, an anorectoplasty, and anoplasty.

Embodiment 205. The non-transitory computer readable medium according to Embodiment 203 or 204, wherein, prior to or after having the operation, the subject has a condition selected from the group consisting of Hirschsprung disease, an anorectal malformation, and a meningocele.

Embodiment 206. The non-transitory computer readable medium according to any one of Embodiments 169-205, wherein an age of the subject is 3 months or older.

Embodiment 207. The non-transitory computer readable medium according to any one of Embodiments 169-206, wherein the module is first performed about 1 week after the operation.

Embodiment 208. The non-transitory computer readable medium according to any one of Embodiments 169-207, wherein the module is performed until the subject is about 20 years old.

Embodiment 209. The non-transitory computer readable medium according to any one of Embodiments 169-208, wherein the one or more first modules are provided to a guardian of the subject, and the subject received the modules indirectly from the non-transitory computer readable medium through the guardian.

Embodiment 210. The non-transitory computer readable medium according to any one of Embodiments 169-209, wherein the one or more first modules consists of the exercise and/or life style module, the sitz bath module, the pelvic splanchnic nerve and/or pelvic muscle exercise module, and the vagal nerve system and/or gastrointestinal (GI) exercise module.

Embodiment 211. The non-transitory computer readable medium according to any one of Embodiments 169-210, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

Embodiment 212. The non-transitory computer readable medium according to any one of Embodiments 169-211, wherein the sensor comprises a touch sensor, and the subject provides the adherence information to the electronic device using the touch sensor.

Embodiment 213. The non-transitory computer readable medium according to any one of Embodiments 169-212, wherein the sensor is a detachable peripheral.

Embodiment 214. The non-transitory computer readable medium according to any one of Embodiments 169-213, wherein the adherence information includes changes of coordinates on at least two points on the subject over time.

Embodiment 215. The non-transitory computer readable medium according to any one of Embodiments 169-214, wherein the adherence information includes changes of coordinates on at least ten points on the subject over time.

Embodiment 216. The non-transitory computer readable medium according to Embodiment 214 or 215, wherein said points are on positions selected from the group consisting of head, shoulder, elbow, wrist, waist, knee, and ankle of the subject.

Embodiment 217. The non-transitory computer readable medium according to any one of Embodiments 214-216, wherein the first instructions comprise changes of co ordinates on said points, and the changes of coordinates of the adherence information are compared with the changes of coordinates of the first instructions.

Embodiment 218. A method for use in an apparatus to rehabilitate a pediatric patient with lower gastrointestinal-operable-congenital anomaly, the method comprising: generating one or more gastrointestinal rehabilitation & cure (GRC) instructions that induce, based on the pediatric patient's performance of the one or more GRC instructions, at least one physiological response rehabilitating bowel and anorectal functions of the pediatric patient after a gastrointestinal anomaly reconstruction operation; and providing the pediatric patient the one or more GRC instructions via the apparatus.

Embodiment 219. The method according to Embodiment 218, wherein the at least one physiological response includes at least one of insulin-like growth factor-1/growth hormone (IGF-1/GH) stimulation, vagal nerve (VN) stimulation, or pelvic splanchnic nerve (PSN) stimulation.

Embodiment 220. The method according to Embodiment 219, wherein the one or more GRC instructions comprise growth hormone (GH) behavior instructions inducing the IGF-1/GH stimulation, vagal nerve (VN) behavior instructions inducing the VN stimulation, and pelvic splanchnic nerve (PSN) behavior instructions inducing the PSN stimulation.

Embodiment 221. The method according to Embodiment 220, wherein the GH behavior instructions comprise sleeping, an unstructured exercise, jumping, stretching, a ball game, a skeletal muscle exercise, an aerobic exercise, and a resistance exercise.

Embodiment 222. The method according to Embodiment 221, further comprising: determining at least one of the GH behavior instructions based on the pediatric patient's growth period.

Embodiment 223. The method according to Embodiment 220, wherein the VN behavior instructions comprise viewing a photo, listening sounds, tactual sensing, gustatory sensing, and olfactory sensing.

Embodiment 224. The method according to Embodiment 220, wherein the PSN behavior instructions comprise warm bathing, cycling walking, hanging, standing upright, touching a ceiling, a freeze game, a movement game, bar hanging, and running.

Embodiment 225. The method according to Embodiment 224, further comprising: determining at least one of the GH behavior instructions based on the pediatric patient's growth period.

Embodiment 226. The method according to Embodiment 225, wherein the pediatric patient's growth period comprises an infant period, a toddler period, a kindergarten period, a grade school period.

Embodiment 227. An apparatus for rehabilitation of a pediatric patient with lower gastrointestinal-operable-congenital anomaly, the apparatus comprising: a processor configured to: generate one or more gastrointestinal rehabilitation & cure (GRC) instructions that induce, based on the pediatric patient's performance of the one or more GRC instructions, at least one physiological response rehabilitating bowel and anorectal functions of the pediatric patient after a gastrointestinal anomaly reconstruction operation; and provide the pediatric patient the one or more GRC instructions.

Embodiment 228. The apparatus of Embodiment 227, wherein the at least one physiological response includes at least one of insulin-like growth factor-1/growth hormone (IGF-1/GH) stimulation, vagal nerve (VN) stimulation, or pelvic splanchnic nerve (PSN) stimulation.

Embodiment 229. The apparatus of Embodiment 228, wherein the one or more GRC instructions comprise growth hormone (GH) behavior instructions inducing the IGF-1/GH stimulation, vagal nerve (VN) behavior instructions inducing the VN stimulation, and pelvic splanchnic nerve (PSN) behavior instructions inducing the PSN stimulation.

Embodiment 230. The apparatus of Embodiment 229, wherein the GH behavior instructions comprise sleeping, an unstructured exercise, jumping, stretching, a ball game, a skeletal muscle exercise, an aerobic exercise, and a resistance exercise.

Embodiment 231. The apparatus of Embodiment 230, wherein the processor is further configured to determine at least one of the GH behavior instructions based on the pediatric patient's growth period.

Embodiment 232. The apparatus of Embodiment 229, wherein the VN behavior instructions comprise viewing a photo, listening sounds, tactual sensing, gustatory sensing, and olfactory sensing.

Embodiment 233. The apparatus of Embodiment 229, wherein the PSN behavior instructions comprise warm bathing, cycling walking, hanging, standing upright, touching a ceiling, a freeze game, a movement game, bar hanging, and running.

Embodiment 234. The apparatus of Embodiment 233, wherein the processor is further configured to determine at least one of the GH behavior instructions based on the pediatric patient's growth period.

Embodiment 235. The apparatus of Embodiment 234, wherein the pediatric patient's growth period comprises an infant period, a toddler period, a kindergarten period, a grade school period.

Embodiment 236. A method according to treating a pediatric patient with lower gastrointestinal-operable-congenital anomaly, the method comprising: providing the pediatric patient one or more gastrointestinal rehabilitation & cure (GRC) instructions that induce, based on the pediatric patient's performance of the one or more GRC instructions, at least one physiological response rehabilitating bowel and anorectal functions of the pediatric patient after a gastrointestinal anomaly reconstruction operation, wherein the at least one physiological response includes at least one of insulin-like growth factor-1/growth hormone (IGF-1/GH) stimulation, vagal nerve (VN) stimulation, or pelvic splanchnic nerve (PSN) stimulation.

Embodiment 237. The method according to Embodiment 236, wherein the one or more GRC instructions comprise growth hormone (GH) behavior instructions inducing the IGF-1/GH stimulation, vagal nerve (VN) behavior instructions inducing the VN stimulation, and pelvic splanchnic nerve (PSN) behavior instructions inducing the PSN stimulation.

1: A method of strengthening or increasing mass of a pelvic floor muscle in a subject, the method comprising:
providing, by an electronic device to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising one or more first instructions for the subject to follow.

2: The method according to claim 1, wherein the electronic device (i) comprises a sensor for sensing adherence by the subject to the first instructions of the one or more first modules, (ii) transmits adherence information, based on the adherence, to a server, and (iii) receives one or more second instructions from the server based on the adherence information.

3: The method according to claim 1, further comprising providing, by the electronic device to the subject, one or more second modules selected from the group consisting of an exercise and/or life style module, a sitz bath module, a pelvic splanchnic nerve and/or pelvic muscle exercise module, and a vagal nerve system and/or gastrointestinal (GI) exercise module, the one or more second modules comprising the one or more second instructions.

4: The method according to claim 3, wherein the one or more first modules or the one or more second modules comprise the exercise and/or life style module, and the exercise and/or life style module comprises one or more first instructions to increase a secretion of at least one of growth hormone (GH) and insulin-like growth factor-1 (IGF-1) in the subject.

5: The method according to claim 1, wherein the exercise and/or life style module comprises one or more first instructions for aerobic, resistance, and/or concurrent exercise.

6: The method according to claim 5, wherein the instructions for the aerobic, the resistance, and/or the concurrent exercise comprise instructions for one or more independently selected from the group consisting of gripping a finger, tummy time, lifting weights, moving toe, moving ear, and a physical activity.

7: The method according to claim 6, wherein the physical activity is selected from the group consisting of a structured exercise, kickball, riding a pedal-driven vehicle, jumping, stretching, playing a ball game, skeletal muscle exercise, walking, and aerobic and resistance exercise.

8: The method according to claim 5, wherein the one or more first instructions comprise instructions for obtaining one or more of deep sleep and adequate sleep.

9: The method according to claim 3, wherein the one or more first modules or the one or more second modules comprise the sitz bath module, and the sitz bath module comprises one or more first instructions to maintain non-ischemic status thereby stimulating myosin formation, stimulate rectal ENS maturation, and/or stimulate GLP2 secretion for rectal endocrine cells.

10: The method according to claim 9, wherein the sitz bath module comprises one or more first instructions to take warm bath, hip bath and/or sitz bath.

11: The method according to claim 3, wherein the one or more first modules or the one or more second modules comprise the pelvic splanchnic nerve and/or pelvic muscle exercise module, and the pelvic splanchnic nerve and/or pelvic muscle exercise module comprises one or more first instructions to stimulate rectal ENS maturation, and/or stimulate GLP2 secretion for rectal endocrine cells.

12: The method according to claim 11, wherein the pelvic splanchnic nerve and/or pelvic muscle exercise module comprises one or more first instructions for cycling away, performing a wheelbarrow, walking, bar hanging, standing upright, trying to touch ceiling, freezing movement, and running.

13: The method according to claim 3, wherein the one or more first modules or the one or more second modules comprise the vagal nerve system and/or GI exercise module, and the vagal nerve system and/or GI exercise module comprises one or more first instructions to stimulate rectal and/or GI ENS maturation, stimulate GLP2 secretion for rectal and/or GI endocrine cells, and/or decrease inflammation signaling factors.

14: The method according to claim 13, wherein the one or more first modules or the one or more second modules comprise the vagal nerve system and/or GI exercise module, and the vagal nerve system and/or GI exercise module comprises one or more first instructions to improve anorectal motility.

15: The method according to claim 14, wherein the one or more first instructions to improve anorectal motility are selected from the group consisting of aerobic exercises including walking, running, cycling, and swimming, walking hand-in-hand with another individual, bar hanging, standing upright, reaching for the ceiling, and limiting motion/playing a freeze game, playing with a small animal, high-intensity playing, adjusting posture, and tapping on a surface.

16: The method according to claim 13, wherein the vagal nerve system and/or GI exercise module comprises at least one instructions selected from the group consisting of sense stimulation instructions for sight, sound, touch, taste, and smell.

17: The method of claim 13, wherein the subject has Inflammatory Bowel Disease (IBS), and a treatment mechanism of the module comprises one or both of anti-inflammation and motility adaptation.

18: The method of claim 13, wherein the subject has chronic constipation, and a treatment mechanism of the module comprises one or more of motility adaptation, biofeedback exercise, and blood circulation.

19: The method of claim 18, wherein the one or more first modules or the one or more second modules further comprise one or both of an anorectal biofeedback module and a sitz bath.

20: The method of claim 13, wherein the subject has Necrotizing EnteroColitis (NEC), and a treatment mechanism of the module comprises anti-inflammation.

21: The method of claim 13, wherein the subject has an Adult-GI-post-operation recovery (anti-emesis/nausea, vomiting, no-gas out), and a treatment mechanism of the module comprises one or both of anti-inflammation and motility adaptation.

22: The method according to claim 1, wherein the subject is a post-operative patient with lower gastrointestinal-operable-congenital anomaly.

23: The method according to claim 22, wherein the subject has had one or more operations selected from the group consisting of a pull-through surgery, an anorectoplasty, and anoplasty.

24: The method according to claim 22, wherein, prior to or after having the operation, the subject has a condition selected from the group consisting of Hirschsprung disease, an anorectal malformation, and a meningocele.

25: The method according to claim 2, wherein the sensor is a detachable peripheral.

26: The method according to claim 25, wherein the adherence information includes changes of coordinates on at least two points on the subject over time.

27: The method according to claim 25, wherein the adherence information includes changes of coordinates on at least ten points on the subject over time.

28: The method according to claim 26, wherein said points are on positions selected from the group consisting of head, shoulder, elbow, wrist, waist, knee, and ankle of the subject.

29: The method according to claim 26, wherein the first instructions comprise changes of coordinates on said points, and the changes of coordinates of the adherence information are compared with the changes of coordinates of the first instructions.

30: A system for strengthening or increasing mass of a pelvic floor muscle in a subject, comprising:
a digital apparatus configured to execute a digital application comprising one or more first modules, for strengthening or increasing mass of a pelvic floor muscle in a subject, wherein the digital apparatus comprises a sensor for sensing adherence by the subject to a first set of instructions of the one or more first modules;
a healthcare provider portal configured to provide one or more options to a healthcare provider to perform one or more tasks to prescribe treatment to strengthen or increase mass of a pelvic floor muscle in the subject based on information received from the digital application; and
an administrative portal configured to provide one or more options to an administrator of the system to perform one or more tasks to manage access to the system by the healthcare provider.

31: A computing system for strengthening or increasing mass of a pelvic floor muscle in a subject in need thereof, comprising:
a display configured to provide, to the subject, one or more first modules selected from the group consisting of an exercise and/or life style module, a sitz bath module, a pelvic splanchnic nerve and/or pelvic muscle exercise module, and a vagal nerve system and/or gastrointestinal (GI) exercise module, each of the one or more first modules comprising one or more first instructions for the subject to follow;
a sensor configured to sense adherence by the subject to the instructions of the one or more first modules;
a transmitter configured to transmit adherence information, based on the adherence, to a server; and
a receiver configured to receive, from the server, one or more second instructions based on the adherence information.
